SEQUENCE LISTING (2 sequences)

SEQ ID NO: 1 - protein, 220 aa, Vaccinia virus
MVYKLVLLFCIASLGYSVEYKNTICPPRQDYRYWYFAAELTIGVNYDINSTIIGECHMSESYIDRNANIVLTGYGLEINMTIMDTDQRFVAAAEGVGKDNKLSVLLFTTQRLDKVHHNISVTITCMEMNCGTTKYDSDLPESIHKSSSCDITINGSCVTCVNLETDPTKINPHYLHPKDKYLYHNSEYGMRGSYGVTFIDELNQCLLDIKELSYDICYRE

SEQ ID NO: 2 - protein, 214 aa, Myxoma virus
MARYIIIVLACLVATSTCATYPKKYWHLAAELTIGLNRYVETVMGECHMKERCDHKTSTLILTGYGLMINITITNVVQRFVAASAGAGDGNKLSIMLFTTHPLTKYSDIYLTITCLEPEGDVGNYGNQLPDSLHHNKDVSITILGSCVTCVNLETNPIKVNPHFTHPISMFVYDNKEDVRGSYGVTFEDELNVCFLDIKKVSYDLCYRQTRYLI

(19) United States
(12) Patent Application Publication
(10) Pub. No.: US 2022/0056481 A1
(43) Pub. Date: Feb. 24, 2022

(54) M2-DEFECTIVE POXVIRUS

(71) Applicant: TRANSGENE SA, ILLKIRCH GRAFFENSTADEN (FR)

(72) Inventors: Patricia KLEINPETER, LAMPERTHEIM (FR); Jean-Baptiste MARCHAND, OBERNAI (FR); Christelle REMY, LOCHWILLER (FR); Doris SCHMITT, PLOBSHEIM (FR)

(73) Assignee: TRANSGENE, ILLKIRCH GRAFFENSTADEN (FR)

(21) Appl. No.: 17/418,426

(22) PCT Filed: Dec. 27, 2019
(86) PCT No.: PCT/EP2019/087063

§ 371 Date: Jun. 25, 2021

(30) Foreign Application Priority Data
Dec. 28, 2018 (EP) 18306874.1
Aug. 21, 2019 (EP) 19306022.5

(51) Int. Cl. C12N 15/86; C07K 16/28; A61P 35/00

(52) U.S. Cl. CPC C12N 15/86; C07K 16/2818; A61P 35/00; C12N 2710/24143; C12N 2710/24122; C12N 2710/24132; C12N 2710/24121

(57) ABSTRACT

The present invention is in the field of oncolytic viruses. The invention provides new poxviruses which are engineered to be defective for the function encoded by the M2L locus (i.e., m2 function). Such poxviruses lack a functional m2 binding activity to at least one or both of CD80 and CD86 co-stimulatory antigens. Said oncolytic poxviruses are preferably vaccinia virus having a total or partial deletion of the M2L locus. The present invention also relates to cells and compositions comprising such poxviruses and their use for treating proliferative diseases such as cancers and for preventing diseases (vaccination, especially in veterinary field). More precisely, the invention provides an alternative to the existing oncolytic viruses which are largely used in virotherapy. The m2-defective poxviruses are particularly useful for the expression of immunomodulatory polypeptides such as anti-CTLA-4 antibodies with the purposes of stimulating or improve immune response.

### TECHNICAL FIELD OF THE INVENTION

The present invention is in the field of oncolytic viruses. The invention provides new poxviruses which are engineered to be defective for the function encoded by the M2L locus (i.e., m2 function). Such poxviruses lack a functional m2 binding activity to at least one or both of CD80 and CD86 co-stimulatory antigens. Said oncolytic poxviruses are preferably vaccinia virus having a total or partial deletion of the M2L locus. The present invention also relates to cells and compositions comprising such poxviruses and their use for treating proliferative diseases such as cancers and for preventing diseases (vaccination, especially in veterinary field). More precisely, the invention provides an alternative to the existing oncolytic viruses which are largely used in virotherapy. The m2-defective poxviruses are particularly useful for the expression of immunomodulatory polypeptides such as anti-CTLA-4 antibodies with the purposes of stimulating or improve immune response.

### BACKGROUND ART

Each year, cancer is diagnosed in more than 12 million subjects worldwide. In industrialized countries, approximately one person out five will die of cancer. Although a vast number of chemotherapeutics exists, they are often ineffective, especially against malignant and metastatic tumors that establish at a very early stage of the disease.

Oncolytic virotherapy has been emerging for two decades based on replication-competent viruses to destroy cancer cells (Russell et al., 2012, Nat. Biotechnol. 30(7): 658-70). Numerous preclinical and clinical studies are presently ongoing to assess in various types of cancers the therapeutic potential of oncolytic viruses armed with a variety of therapeutic genes.

Therapeutic genes are usually inserted in the viral genome within non-essential genes to retain oncolytic phenotype. Insertion in the J2R locus (tk) is widely used in the art insofar as it also facilitates identification of recombinant virus in the presence of BUdR (Mackett et al., 1984 J. of Virol., 49: 857-64; Boyle et al., 1985, Gene 35, 169-177). However, other loci have been also proposed, e.g. into Hind F fragment, into M2L locus (Smith et al., 1993, Vaccine 11(1): 43-53; Guo et al., 1990, J. Virol. 64: 2399-2406; Bloom et al., 1991, J. Virol. 65(3): 1530-42; Hodge et al., 1994, Cancer Res. 54: 5552-5; McLaughlin et al., 1996, Cancer Res. 56: 2361-67) and A56R locus (encoding hemagglutinin (HA)).

Poxviruses and especially Vaccinia viruses (VV) have provided several promising oncolytic candidates (De Graaf et al., 2018, doi.org/10.1016/j.cytogfr.2018.03.006), such as JX594 (Sillajen/Transgene), GL-ONC1 (Genelux), TG6002 (Transgene) and vvDD-CDSR (University of Pittsburgh). These oncolytic VV originate from different VV strains with diverse genomic modifications and expression of various therapeutic genes. JX-594 (Wyeth strain) attenuated through deletion of the viral J2R gene (which encodes thymidine kinase (tk)) and further armed with GM-CSF is currently under clinical evaluation in a randomized Phase III trial in hepatocellular carcinoma (Parato et al., 2012, Molecular Therapy 20(4): 749-58). GL-ONC1 was generated by inserting three expression cassettes respectively in place of the F14.5L, J2R and A56R loci of the parental viral Lister strain genome. On the same line, TG6002, a J2R (tk) and 14L (14L locus encodes ribonucleotide reductase (rr-))-defective VV (Copenhagen strain) encoding the FCU1 enzyme that converts the non-toxic 5-fluorocytosine (5-FC) into the cytotoxic 5 fluorouracile (5-FU) is being evaluated in some clinical trials. The tk and rr double deletion restricts the replication of the virus to cells containing a high pool of nucleotides, making TG6002 unable to replicate in resting cells (Foloppe et al., 2008, Gene Ther. 15: 1361-71; WO2009/065546). vvDD-CDSR is currently assayed in patients with refractory cutaneous and subcutaneous tumors. It was engineered by double deletion of the tk (J2R locus) and vaccinia growth factor (vgf) encoding genes and armed with both a cytosine deaminase (CD) gene for conversion of 5-FC to 5-FU and a somatostatin receptor (SR) gene for in vivo imaging.

Initially, direct oncolysis was thought to be the sole mechanism through which oncolytic viruses exert their antitumor effect. Only recently, it was appreciated that the immune system plays a critical role in the success of virotherapy (Chaurasiya et al., 2018, Current Opinion in Immunology 51: 83-90). However, most viruses have developed self-defense mechanisms through a repertoire of proteins involved in immune evasion and immune modulation aimed at blocking many of the strategies employed by the host to combat viral infections (Smith and Kotwal, 2002, Crit. Rev. Microbiol. 28(3): 149-85). Moreover, tumor cells have also evolved a mechanism of T cell exhaustion to escape host's immune system, which is characterized by the upregulation of inhibitory receptors; CTLA-4 (for cytotoxic T-lymphocyte associated protein-4; also known as CD152) and PD-1 (for programmed cell death protein 1) and its ligands PD-L1 and PD-L2, being the most documented. These immunosuppressive receptors serve as immune checkpoints acting at different levels of T cell immunity. CTLA-4 inhibits early stages of T cell activation in the lymph node and also stimulates undesirable Treg while PD-1 acts at a later stage.

More specifically, activation of T cells involves the interaction of co-stimulatory ligands such as CD80 (also designated B7-1) and CD86 (also designated B7.2), present at the surface of the APC (for Antigen Presenting cell) with receptors present at the surface of T cells such as CD28, CTLA-4 and PDL-1. CD80 is the ligand for these 3 cell surface receptors whereas CD86 binds CD28 and CTLA-4. CD28 receptor is constitutively expressed on resting T cells and ligation of CD28 with costimulatory CD80 and CD86 ligands delivers a positive stimulatory signal to T cells, induces them to proliferate and secrete IL-2 and inhibits apoptosis through increased expression of Bcl-XL (Chen, 2004, Nat. Rev. Immunol. 4: 336-347). In contrast, CTLA-4 or PD-L1 play a role in negative regulation of T cells either following initial T cell activation (for CTLA-4) or at a later stage (for PD-L1). Specifically, upon ligation with CD80 and CD86 costimulatory ligands, CTLA-4 acts in cis on activated T cells to oppose the co-stimulatory signal provided by interactions of CD28 with CD80 and CD86 and is involved in IL-10 production. In addition, CTLA-4 is constitutively expressed on a subset of immunosuppressive regulatory T cells (Treg). On the other hands, ligation of CD80 to PD-L1 on the surface of the T regulatory cells have been demonstrated to increase the proliferation of these immunosuppressive cells (Yi, 2011, J Immunol. 186:2739-2749). CTLA4 was identified in 1987 (Brunet et al., 1987, Nature 328: 267-70) and is encoded by the CTLA4 gene (Dariavach et al., Eur. J. Immunol. 18: 1901-5). The complete CTLA-4 nucleic acid sequence can be found under GenBank Accession No LI 5006.

There has been increasing interest in blocking such immunosuppressive checkpoints as a means of rescuing exhausted antitumor T cells. A vast number of antagonistic antibodies have been developed during the last decade (Kahn et al., 2015, J. Oncol. Doi: 10.1155/2015/847383) and several have been approved by the FDA of which the first were against CTLA4 (e.g. Ipilimumab/Yervoy, Bristol-Myers Squibb) and PD-1 (pembrolizumab/Keytruda developed by Merck and Nivolumab/Optivo developed by BMS). While conventional treatments rely on the administration of the antibodies to the patients, vectorization by virus or plasmid vectors is now being considered to deliver these antibodies directly to tumor cells (see e.g. WO2016/008976). For example, a tk- and rr-VV armed with anti-PD-1 was shown to induce tumor growth control in MCA-205 mouse model (Kleinpetter et al., 2016, OncoImmunology 5(10): e1220467).

However, due to the complex nature of these immunity-interacting molecules and virus vectors and the risk of triggering cascade events, preclinical and even more clinical studies may be difficult to implement.

Therefore, there is still a need to further develop oncolytic viruses, compositions and methods for delivering therapeutic polypeptides such as checkpoint-directed antagonist antibodies for enhancing anti-tumoral adaptative immune responses in cancerous patients.

### Technical Problem and Proposed Solution

Unexpectedly, the inventors have identified that supernatants of cells infected with vaccinia virus (VV) interact with the co-stimulatory CD80 and CD86 ligands whereas supernatants of cells infected with the attenuated Modified Vaccinia virus Ankara (MVA) lack this property. The inventors have assigned the CD80 and CD86 binding properties to the M2 protein encoded by the VV M2L locus. Before the invention, M2 was reported as a protein retained in endoplasmic reticulum acting as an inhibitor of the NfKb pathway (Hinthong et al., 2008, Virology 373(2): 248-62) and involved in uncoating of the virus (Baoming Liu et al., 2018, J. Virol. 92(7) e02152-17). Further to VV, the inventors have identified the existence of M2 orthologs in numerous replicative poxviruses.

The present invention illustrates the capacity of the M2 protein of binding to CD80 and CD86 and impacting three immunosuppressive pathways; respectively i) it blocks the CD80 and CD86 interactions with CD28; ii) it promotes the interaction of CD80 with PD-L1; and iii) it triggers a reverse signalling to the CD80/CD86 positive cells.

In the context of the present invention, the inventors have generated a vaccinia virus that is defective for m2 function. When armed with an immunomodulatory polypeptide such as an anti-CTLA-4 antibody, its expression inhibits the CTLA-4-mediated immunosuppressive signals and it is expected that the absence of m2 permits to redirect the T cell response to the CD28-mediated immunostimulatory signals whereas a M2L-positive vaccinia virus would negatively interfere with such CD28-mediated positive signals due to the m2 binding to CD80 and CD86 co-stimulatory ligands.

Importantly and surprisingly poxviruses described herein are expected to stimulate or improve immune response, especially the lymphocyte-mediated response, against an antigen due to the absence of synthesis of a functional m2 protein in the infected cells whereas in a conventional poxvirus (M2L-positive), the produced viral m2 protein would bind CD80 and CD86 co-stimulatory ligands and, thus, prevent CD28-mediated positive pathways. Moreover, poxviruses described herein display an enhanced propensity to be accepted by the host's immune system since they lack a protein involved in immune evasion of the virus; which feature provides a competitive advantage over M2-positive poxviruses. The present invention offers a unique product combining oncolysis for killing dividing cells and immunostimulatory activities, e.g. for breaking cancer-associated immune exhaustion, thus improving therapeutic capacities of the oncolytic virus.

This technical problem is solved by the provision of the embodiments as defined in the claims. Other and further aspects, features and advantages of the present invention will be apparent from the following description of the presently preferred embodiments of the invention. These embodiments are given for the purpose of disclosure.

### SUMMARY OF THE INVENTION

The disclosure relates to poxviruses, especially oncolytic poxviruses, that have been engineered to be defective for the m2 protein encoded by the M2L locus, and methods of generating and using such viruses. As disclosed herein, poxviruses defective for the m2 function encoded by the M2L locus, optionally in combination with other functional inactivation(s) of the tk-encoding locus and/or rr-encoding locus were generated and isolated. m2-defective vaccinia virus engineered to express an anti-CTLA4 antibody are also comtemplated.

According to a first aspect of the present invention, there is provided a modified poxvirus which genome comprises in the native (wild-type) context a M2L locus encoding a functional m2 poxviral protein and which is modified to be defective for the said m2 function; wherein said functional m2 poxviral protein is able to bind CD80 or CD86 co-stimulatory ligands or both CD80 and CD86 co-stimulatory ligands and wherein said defective m2 function is unable to bind said CD80 and CD86 co-stimulatory ligands.

In one embodiment, the modified poxvirus is generated or obtained from a Chordopoxvirinae, preferably selected from the group of genus consisting of Avipoxvirus, Capripoxvirus, Leporipoxvirus, Molluscipoxvirus, Orthopoxvirus, Parapoxvirus, Suipoxvirus, Cervidpoxvirus and Yatapoxvirus. In a preferred embodiment, said modified poxvirus is a member of the Orthopoxvirus, preferably selected from the group consisting of vaccinia virus (VV), cowpox (CPXV), raccoonpox (RCN), rabbitpox, Monkeypox, Horsepox, Volepox, Skunkpox, variola virus (or smallpox) and Camelpox; with a specific preference for a modified vaccinia virus.

In one embodiment, the inability to bind said CD80 and CD86 co-stimulatory ligands originates from a genetic lesion within the M2L locus or from an abnormal interaction impairing the m2 function either directly or indirectly. Said genetic lesion(s) include partial or total deletion and/or one or more non-silent mutation(s) (that translates in a change of amino acid residue(s)) either within the m2-coding sequence or in the regulatory elements controlling M2L expression, preferably leading to the synthesis of a defective m2 protein or to the lack of m2 synthesis. Said genetic lesion is preferably a partial or entire deletion of the M2L locus.

In one embodiment, the modified poxvirus is further modified in a region other than M2L locus; in particular in the J2R locus (resulting in a modified poxvirus defective for both m2 and tk functions) or in the 14L/F4L locus/loci (resulting in a modified poxvirus defective for both m2 and rr functions). Preferably, the modified poxvirus is further modified in the J2R and 14L/F4L loci, resulting in a modified poxvirus defective for m2, tk and rr activities.

In one embodiment, the modified poxvirus is oncolytic.

In one embodiment, said modified poxvirus is recombinant. Said modified poxvirus is preferably engineered to express at least one polypeptide selected from the group consisting of antigenic polypeptides, polypeptides having nucleos/tide pool modulating function and immunomodulatory polypeptides. Said immunomodulatory polypeptide is desirably selected from the group consisting of cytokines, chemokines, ligands and antibodies or any combination thereof. In a preferred embodiment, said modified poxvirus is defective for m2, tk and rr activities and encodes an anti-CTLA-4 antibody. In another preferred embodiment, said modified poxvirus is defective for m2, tk and rr activities and encodes an anti-PD-L1 antibody.

According to another aspect, there is provided a method for producing the modified poxvirus comprising the steps of a) preparing a producer cell line, b) transfecting or infecting the prepared producer cell line with the modified poxvirus, c) culturing the transfected or infected producer cell line under suitable conditions so as to allow the production of the virus, d) recovering the produced virus from the culture of said producer cell line and optionally e) purifying said recovered virus.

According to a further aspect, there is provided a composition comprising a therapeutically effective amount of the modified poxvirus and a pharmaceutically acceptable vehicle. The composition desirably comprises from approximately 10³ to approximately 10¹² pfu, advantageously from approximately 10⁴ pfu to approximately 10¹¹ pfu, preferably from approximately 10⁵ pfu to approximately 10¹⁰ pfu; and more preferably from approximately 10⁶ pfu to approximately 10⁹ pfu of the modified poxvirus. The composition is preferably formulated for intravenous or intratumoral administration.

In still another aspect, the composition is for use for treating or preventing a proliferative disease selected from the group consisting of cancers as well as diseases associated to an increased osteoclast activity such as rheumatoid arthritis and osteoporosis and cardiovascular diseases such as restenosis. The cancer to be treated or prevented is preferably selected from the group consisting of renal cancer, prostate cancer, breast cancer, colorectal cancer, lung cancer, liver cancer, gastric cancer, bile duct carcinoma, endometrial cancer, pancreatic cancer and ovarian cancer. The modified poxvirus and composition is for use as stand-alone therapy or in conjunction with one or more additional therapies, preferably selected from the group consisting of surgery, radiotherapy, chemotherapy, cryotherapy, hormonal therapy, toxin therapy, immunotherapy, cytokine therapy, targeted cancer therapy, gene therapy, photodynamic therapy and transplantation.

In still another aspect, the modified poxvirus or composition is for use for stimulating or improving an immune response.

### DESCRIPTION OF THE FIGURES

FIG. 1 illustrates CD80/CTLA4 (1A) and CD86/CTLA4 (1B) competition ELISA assays carried out with the supernatants collected from avian DF1 cells either uninfected (dotted line) or infected with wild type VV (diamond) or Yervoy (inverted triangle). Binding of His-tagged B7-Fc proteins to immobilized CTLA4-Fc was performed using an anti-His tag-HRP conjugated antibody.

FIG. 2 illustrates CD80/CTLA4 competition ELISA carried out with the supernatants collected from HeLa cells infected with MVA (MVA), vaccinia virus of Copenhagen strain (Cop VV), Western Reserve strain (WR VV), Wyeth strain (Wyeth VV), raccoonpox (RCN), rabbitpox (RPX), cowpox (CPX), fowlpox (FPV) and pseudocowpox (PCPV) and the supernatant of uninfected HeLa cells (negative control).

FIG. 3 illustrates western blot performed in non-reducing SDS-PAGE with supernatants of CEF cells either uninfected (Sup.cells) or infected with MVA (Sup.MVA) or Copenhagen vaccinia virus (Sup.VV) collected directly or 20-fold concentrated (×20) and probed with fusions of human CD86 with Fc fragment (hCD86-Fc), human CD80 with Fc fragment (hCD80-Fc) and human CTLA4 with Fc fragment (hCTLA4-Fc). Detection was performed with an anti-Fc conjugated antibody.

FIG. 4 illustrates competition ELISA testing the interaction of biotinylated-CD80 and biotinylated-CD86 with their cognate receptors, CD28/CD86, CD28/CD80, CTLA4/CD80 and PDL1/CD80 respectively. Supernatants collected from CEF cells infected with MVA (MVA) and vaccinia virus of Copenhagen strain (VV) are compared to the supernatant of uninfected CEF cells (CEF) (negative control) and Yervoy antibody (10 μg/ml). Reactivity of recombinant human PD1 (hPD1), human CD80 (hCD80) and human CTLA4 (hCTLA4) all at 10 μg/ml are used as positive control for competing with the PDL1/CD80 interaction. Detection of the bound biotinylated B7 proteins was performed using HRP conjugated streptavidin.

FIG. 5A illustrates the experimental approach used to identify the “interference factor (IF)” by affinity chromatography with immobilized CD86-Fc fusion and FIG. 5B provides the sequence of the IF captured in VV-infected CEF cells.

FIG. 6 illustrates CD80/CTLA4 competition ELISA carried out with the supernatants collected from uninfected HeLa or DF1 cells (HeLa or DF1) as negative controls or infected with a double deleted (tk-rr-) Copenhagen vaccinia virus (VVTG18277) or a triple deleted (tk-rr-m2-) Copenhagen vaccinia virus (COPTG19289). Binding of his-tagged CD80-Fc proteins to immobilized CTLA4-Fc was monitored using an anti-His tag-HRP conjugated antibody.

FIG. 7 illustrates oncolytic activity of the tk-rr-m2-vaccinia virus (COPTG19289) and its tk-rr-counterpart (VVTG18277) four days after infection of LOVO (A) and HCT116 (B) cells at various MOI (from 10⁻¹ to 10⁻⁴). MOCK-treated cells are used as negative control.

FIG. 8 illustrates luciferase expression in C57BL/6 mice subcutaneously implanted with B16F10 tumors. VVTG18277 virus and COPTG19289 (10⁷ pfu) were injected intratumorally at day 0, 3, 6, 10 and 14 and tumor samplings were collected at day 1, 2, 6, 9, 13 and 16 for evaluation of luciferase activity par gram of tumor (RLU/g tumor). Three mice were included by time point.

FIG. 9 illustrates antitumoral activity in Balb/c mice subcutaneously implanted with CT26 tumors. 10⁷ pfu of VVTG18277 (square), COPTG19289 (triangle) or Mock (circle) were injected intratumorally at D0, D3, D6, D10 and D14 (10 mice/group). Tumor growth was followed twice a week (mice were killed when the tumor volume reached 2000 mm³).

FIG. 10 illustrates antitumoral activity in Swiss Nude mice subcutaneously implanted with HT116 tumors. Mice (10 mice/group) received a single intravenously injection at D10 when tumor reached 100 to 200 mm3 of either 10⁵ (A) or 10⁷ (B) pfu of VVTG18277 (circle), COPTG19289 (square) or Mock (diamond). Tumor growth was followed twice a week.

FIG. 11 illustrates the effect of supernatant of cells infected by M2 defective poxvirus on mixed lymphocyte reaction (MLR). PBMC were purified from two different donors and cultured in the presence of supernatants obtained from CEF infected (MOI 0.05) with COPTG19289 (tk-, rr- and m2-), VVTG18058 (tk-rr-) or MVAN33 (wild type). Culture supernatants were harvested 48 h post-infection and concentrated about 20-fold. These concentrated supernatants were added to the PBMC culture (20 μL in 200 μL) either undiluted or diluted 10 or 100-fold to yield a final “supernatant concentration” of 2, 0.2 and 0.02-fold, respectively. The amount of IL-2 secreted in the culture medium of PBMC was measured by ELISA. IL-2 measurement was made in triplicate for each sample tested. The measures were normalized by dividing the mean of IL-2 concentration of the three replicates of a given sample by the mean of IL-2 concentration of the three replicates of PBMC incubated with medium.

FIG. 12 illustrates the effect on tumor volume provided by the M2 defective COPTG19289 in a humanized mouse model. NOD/Shi-scid/IL-2Rγnull immunodeficient mice (NCG) were humanized with CD34+ human stem cells and engrafted with human colorectal carcinoma cells HCT-116 (5×10⁶ cells injected SC in one mouse's flank; representing D0). Twelve days post implantation (D12), mice received a single IV injection of either COPTG19289 (TD) or the m2+ counterpart VVTG18058 (DD) at doses of 10⁶ pfu (A) or 10⁵ pfu (B). Vehicle-treated mice were used as negative controls. Tumor growth were monitored over 60 days post cell implantation. Mean tumor growth in mm³ is represented for each group as a function of the number of days post cell injection.

FIG. 13 illustrates the effect on survival provided by the M2 defective COPTG19289 in the humanized NCG-CD34+ mouse model described above. Twelve days post tumor implantation (D12), mice received a single IV injection of either COPTG19289 (TD) or the m2+ counterpart VVTG18058 (DD) at doses of 10⁶ pfu (A) or 10⁵ pfu (B). Vehicle-treated mice were used as negative controls. Mice survival were monitored over 90 days post cell implantation. Survival (percent) is given for each group as a function of the number of days post cell injection.

### DETAILED DESCRIPTION

### General Definitions

A number of definitions are provided here that will assist in the understanding of the invention. However, unless otherwise defined, all technical and scientific terms used herein have the same meaning as commonly understood by one of ordinary skill in the art to which this invention belongs. All references cited herein are incorporated by reference in their entirety.

As used throughout the entire application, the terms “a” and “an” are used in the sense that they mean “at least one”, “at least a first”, “one or more” or “a plurality” of the referenced components or steps, unless the context clearly dictates otherwise. For example, the term “a cell” includes a plurality of cells, including mixtures thereof.

The term “one or more” refers to either one or a number above one (e.g. 2, 3, 4, 5, etc).

The term “and/or” wherever used herein includes the meaning of “and”, “or” and “all or any other combination of the elements connected by said term”.

The term “about” or “approximately” as used herein means within 20%, preferably within 10%, and more preferably within 5% of a given value or range.

As used herein, when used to define products, compositions and methods, the term “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are open-ended and do not exclude additional, unrecited elements or method steps. Thus, a polypeptide “comprises” an amino acid sequence when the amino acid sequence might be part of the final amino acid sequence of the polypeptide. “Consisting of” means excluding other components or steps of any essential significance. Thus, a composition consisting of the recited components would not exclude trace contaminants and pharmaceutically acceptable carriers. A polypeptide “consisting of” an amino acid sequence refers to the presence of such an amino acid sequence with optionally only a few additional and non-essential amino acid residues. It is nevertheless preferred that the polypeptide does not contain any amino acids but the recited amino acid sequence. In the present description, the term “comprising” (especially when referring to a specific sequence) may be replaced with “consisting of”, if required.

Within the context of the present invention, the terms “nucleic acid”, “nucleic acid molecule”, “polynucleotide” and “nucleotide sequence” are used interchangeably and define a polymer of any length of either polydeoxyribonucleotides (DNA) (e.g. cDNA, genomic DNA, plasmids, vectors, viral genomes, isolated DNA, probes, primers and any mixture thereof) or polyribonucleotides (RNA) (e.g. mRNA, antisense RNA, SiRNA) or mixed polyribo-polydeoxyribonucleotides. They encompass single or double-stranded, linear or circular, natural or synthetic, modified or unmodified polynucleotides.

The term “polypeptide” is to be understood to be a polymer of at least nine amino acid residues bonded via peptide bonds regardless of its size and the presence or not of post-translational components (e.g. glycosylation). No limitation is placed on the maximum number of amino acids comprised in a polypeptide. As a general indication, the term refers to both short polymers (typically designated in the art as peptide) and to longer polymers (typically designated in the art as polypeptide or protein). This term encompasses native polypeptides, modified polypeptides (also designated derivatives, analogs, variants or mutants), polypeptide fragments, polypeptide multimers (e.g. dimers), fusion polypeptides among others. The term also refers to a recombinant polypeptide expressed from a polynucleotide sequence which encodes said polypeptide. Typically, this involves translation of the encoding nucleic acid into a mRNA sequence and translation thereof by the ribosomal machinery of the cell to which the polynucleotide sequence is delivered.

The term “identity” refers to an amino acid to amino acid or nucleotide to nucleotide correspondence between two polypeptide or nucleic acid sequences. The percentage of identity between two sequences is a function of the number of identical positions shared by the sequences, taking into account the number of gaps which need to be introduced for optimal alignment and the length of each gap. Various computer programs and mathematical algorithms are available in the art to determine the percentage of identity between amino acid sequences, such as for example the Blast program available at NCBI or ALIGN in Atlas of Protein Sequence and Structure (Dayhoffed, 1981, Suppl., 3: 482-9), or the algorithm of Needleman and Wunsh (J. Mol. Biol. 48,443-453, 1970). Programs for determining identity between nucleotide sequences are also available in specialized data base (e.g. Genbank, the Wisconsin Sequence Analysis Package, BESTFIT, FASTA and GAP programs). Those skilled in the art can determine appropriate parameters for measuring alignment including any algorithms needed to achieve maximum alignment over the sequences to be compared. For illustrative purposes, “at least 70%” means 70% or above (including 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% whereas “at least 80% identity” means 80% or above (including 81%, 82%, 83%, 84%, 85%, 86%, 87%, 88%, 89%, 90%, 91%, 92%, 93%, 94%, 95%, 96%, 97%, 98%, 99% or 100% and “at least 90%” 90%, 91%, 92%, 93%, 94%, 95%, 96%, 97%, 98%, 99% or 100%).

As used herein, the term “isolated” refers to a component (e.g. polypeptide, nucleic acid molecule, virus, vector, etc.), that is removed from its natural environment (i.e. separated from at least one other component(s) with which it is naturally associated or found in nature). For example, a nucleotide sequence is isolated when it is separated of sequences normally associated with it in nature (e.g. dissociated from a genome) but it can be associated with heterologous sequences.

The term “obtained from”, “originating” or “originate” and any equivalent thereof is used to identify the original source of a component (e.g. polypeptide, nucleic acid molecule, virus, vector, etc.,) but is not meant to limit the method by which the component is made which can be, for example, by chemical synthesis or recombinant means.

As used herein, the term “host cell” should be understood broadly without any limitation concerning particular organization in tissue, organ, or isolated cells. Such cells may be of a unique type of cells or a group of different types of cells such as cultured cell lines, primary cells and dividing cells. In the context of the invention, the term “host cells” preferably refers to eukaryotic cells such as mammalian (e.g. human or non-human) cells as well as cells capable of producing the poxvirus described herein. This term also includes cells which can be or has been the recipient of the poxvirus as well as progeny of such cells.

The term “subject” generally refers to an organism for whom any poxvirus, composition and method described herein is needed or may be beneficial. Typically, the organism is a mammal, particularly a mammal selected from the group consisting of domestic animals, farm animals, sport animals, and primates. Preferably, the subject is a human who has been diagnosed as having or at risk of having a proliferative disease such as a cancer. The terms “subject” and “patients” may be used interchangeably when referring to a human organism and encompasses male and female. The subject to be treated may be a newborn, an infant, a young adult, an adult or an elderly.

The term “treatment” (and any form of treatment such as “treating”, “treat”) as used herein encompasses prophylaxis (e.g. preventive measure in a subject at risk of having the pathological condition to be treated) and/or therapy (e.g. in a subject diagnosed as having the pathological condition), eventually in association with conventional therapeutic modalities. The result of the treatment is to slow down, cure, ameliorate or control the progression of the targeted pathological condition. For example, a subject is successfully treated for a cancer if after administration of a poxvirus as described herein, the subject shows an observable improvement of its clinical status.

The term “administering” (or any form of administration such as “administered”) as used herein refers to the delivery to a subject of a therapeutic agent such as the poxvirus described herein.

The term “combination” or “association” as used herein refers to any arrangement possible of various components (e.g. a poxvirus and one or more substance effective in anticancer therapy). Such an arrangement includes mixture of said components as well as separate combinations for concomitant or sequential administrations. The present invention encompasses combinations comprising equal molar concentrations of each component as well as combinations with very different concentrations. It is appreciated that optimal concentration of each component of the combination can be determined by the artisan skilled in the art.

M2-Defective Poxvirus

In one aspect, the present invention provides a modified poxvirus which genome comprises in the native (wild-type) context a M2L locus encoding a functional m2 poxviral protein and which is modified to be defective for the said m2 function; wherein said functional m2 poxviral protein is able to bind CD80 or CD86 co-stimulatory ligands or both CD80 and CD86 co-stimulatory ligands and wherein said defective m2 function is unable to bind said CD80 and CD86 co-stimulatory ligands.

As used herein, the term “poxvirus” or “poxviral” refers to any Poxviridae virus identified at present time or being identified afterwards that is infectious for one or more mammalian cells (e.g. human cells) and which genome comprises in the native (i.e. wild-type) context a M2L locus encoding a functional so-called M2 protein. The term “virus” as used in the context of poxvirus or any other virus mentioned herein encompasses the viral genome as well as the viral particle (encapsided and/or enveloped genome).

Poxviruses are a broad family of DNA viruses containing a double-stranded genome. Like most viruses, poxviruses have developed self-defense mechanisms through a repertoire of proteins involved in immune evasion and immune modulation aimed at blocking many of the strategies employed by the host to combat viral infections (Smith and Kotwal, 2002, Crit. Rev. Microbiol. 28(3): 149-85). Typically, the poxvirus genome encodes more than 20 host response modifiers that allow the virus to manipulate host immune responses and, thus, facilitate virus replication, spread, and transmission. These include growth factors, anti-apoptotic proteins, inhibitors of the NFkB pathway and interferon signalling, and down-regulators of the major histocompatibility complex (MHC).

For general guidance, the wild type vaccinia virus (VV) genome comprises a M2L locus which coding sequence encodes a protein called m2 produced during the early stage of the virus life cycle. It is either secreted or localized in the reticulum endoplasmic (RE) and likely glycosylated (Hinthong et al., 2008, Virology 373: 248-262). Although its function is still under investigation, it is involved in core uncoating and viral DNA replication (Liu et al., 2018, J. Virol., doi/10.1128/JVI.02152-17) but it is dispensable for in vitro viral replication (Smith, 1993, Vaccine 11: 43-53). In addition, its function of downregulating the cellular NF-κB transcription factor via Erk1 phosphorylation inhibition is now well established (Gedey et al., 2006, J. Virol. 80: 8676-85) suggesting that m2 is thus involved in the host's antiviral response during poxviral infection. The VV “M2L” locus is present in the 5′ third part of the wild-type VV genome; specifically, the coding sequence is located between position 27324 and position 27986 of the Copenhagen (Cop) VV genome. The Cop M2L-encoded gene product is a protein of 220 amino acids (having the amino acid sequence shown in SEQ ID NO: 1; also disclosed in Uniprot under P21092 accession number) and composed of a mature polypeptide long of 203 amino acid residues including 8 Cys residues and a N-term 17 amino acid residue long signal peptide also having one Cys residue.

The poxvirus genome in the native context is a double-stranded DNA of approximately 200 kb and has the potential of encoding nearly 200 proteins with different functions including a M2L locus. The genomic sequence and the encoded open reading frames (ORFs) are well known. The modified poxvirus of the invention comprises a genome which has been modified by the man's hands to be at least defective for the m2 function encoded by a native M2L locus and may further comprise one or more additional modifications such as those described herein.

Identification of the Presence of a M2L Locus within a Poxviral Genome

Determination if a given poxvirus comprises or not in the native context a M2L locus encoding a functional m2 protein is within the reach of the skilled artisan using the information given herein and the general knowledge in the art. The particular choice of assay technology is not critical and it is within the reach of those skilled in the art to adapt any of these conventional methodologies to the determination if a candidate poxvirus comprises a M2L locus encoding a functional m2 protein.

In one embodiment, a M2L locus can be identified in a given poxvirus by hybridization or PCR techniques using the information given herein and designing appropriate probes or primers to screen the poxviral genomic sequence. For general guidance, hybridization assays are typically based on oligonucleotide probes derived from the known nucleotide (nt) sequence information set forth herein for M2L locus to be detected with nucleic acids extracted from cells infected or containing such a candidate poxvirus, under conditions suitable for hybridization. Oligonucleotide probe is a short piece of single-stranded RNA or DNA (usually 10 to 30 nucleotides long) that is designed to be complementary (i.e. at least 80% identity) to the target M2L sequence. Probes are preferably labeled to permit detection (e.g. a radioactively, fluorescently or enzymatically-labeled probes). Hybridization is usually performed under stringent conditions allowing only specific hybrids to be formed.

In still another or alternative embodiment, the presence of a M2L locus in the genome of a given poxvirus can be identified based on the amino acid sequence of the encoded gene product. For example, the presence of a M2L locus can be identified by translational analysis of the genomic sequence and blasting the amino acid sequences of the encoded open reading frames (ORFs) in available databases against the known poxviral m2 proteins such as the Cop VV m2 (SEQ ID NO: 1) or the myxoma virus gp-120 like protein (SEQ ID NO: 2) to search for the presence of an encoded ORF displaying at least 40%, desirably at least 50%, preferably, at least 70%, more preferably at least 80% and as an absolute preference at least 90% sequence identity with the amino acid sequence shown in SEQ ID NO: 1 or in SEQ ID NO: 2.

Alternatively or in addition, the amino acid sequences of the ORFs encoded by the poxviral genome can be aligned against available databases. The candidate poxvirus is considered as comprising a M2L locus if it encodes a so-called m2 polypeptide family which gives an outcome after search in domain databases (e.g, Gene3D, PANTHER, Pfam, PIRSF, PRINTS, ProDom, PROSITE, SMART, SUPERFAMILY or TIGRFAMs) which is the same as the outcome of the m2 VV protein (referenced in Uniprot under accession number P21092; also disclosed herein as SEQ ID NO: 1). Therefore, a candidate poxvirus is identified as comprising a M2L locus if it encodes a polypeptide which, when submitted to a Blast analysis using the above-cited databases, is assigned in Uniprot a PFAM motif n° PF04887 or an Interpro motif n° IPR006971 signature.

Functionality of the Encoded m2 Protein.

A functional m2 protein as used herein refers to the capacity of said protein of binding CD80 and/or CD86 co-stimulatory ligands either in vitro or in vivo. The ability of a poxvirus to encode a functional m2 polypeptide can be evaluated by routine techniques. Standard assays to evaluate the binding ability of a protein to its target are known in the art, including for example, Biacore™, calorimetry, fluorometry, Bio-Layer Interferometry, Immunoblot (e.g. Western blot), RIAs, flow cytometry and ELISAs. The particular choice of assay technology is not critical and it is within the reach of those skilled in the art to adapt any of these conventional methodologies to determine if a candidate m2 protein binds to CD80 and/or CD86 co-stimulatory ligands.

For example, supernatants of cells infected with the candidate poxvirus can be used to probe CD80 or CD86 either immobilized on plate (ELISA) or displayed on cell surface (FACS). Sandwich competition ELISA assays (see the Example section) are particularly appropriate due to the fact that there is no need to generate a tagged recombinant protein to get a result. For example, ELISA plates may be coated with a ligand of interest (e.g. CD86-Fc) before adding the sample to be tested (e.g. a supernatant of cells infected with a poxvirus). If the sample comprises a M2 polypeptide, it will bind to the coated ligand. Then, a detection ligand is added which is usually labelled to be detected, e.g. by the action of an enzyme that converts the labelling substance into a coloured product which can be measured using a plate reader (e.g. CTLA4-Fc with a Histag recognized by anti-Histag antibodies coupled to HRP (for horseradish peroxidase). A reduction of chromogenic detection in the presence of a candidate sample as compared to no sample or a negative control sample is indicative that the sample contains a M2 polypeptide competing with the detection ligand for binding to the coated ligand. One may also proceed vice versa, e.g. by using CTLA-4-Fc as coated ligand and CD80-Fc-Histag as detection ligand.

“Defective for m2 function” as used herein is intended to mean the inability of a m2 protein to bind CD80 and/or CD86 co-stimulatory ligands either in vitro or in vivo. This inability may originate from a genetic lesion within the native M2L locus that prevents the normal binding activity of the encoded m2 protein. Thus, functional inactivation could result from one or more mutation(s) in the M2L locus. Such a mutation is preferably selected from the group consisting of insertions, deletions, and base changes in either the coding sequence or in the regulatory sequences controlling expression of the m2 protein. Alternatively, functional inactivation may occur by the abnormal interaction of the m2 protein with one or more other gene products which bind to or otherwise prevent the functional activity of said m2 protein.

For general guidance, the inventors have indeed identified a M2L locus (encoding a functional m2 protein or ortholog thereof) in a vast variety of poxviruses as described hereinafter; more specifically in seven strains of vaccinia virus, in seven strains of myxoma virus, in 4 strains of Monkeypox, in multiple strains of cowpox virus, in eight strains of variola virus as well as in a variety of other poxviruses including, but not limited to, Horsepox, Taterapox, Camelpox, Raccoonpox, Shunkpox, Yokapox, Rabbit fibroma virus, Murmansk pox, Eptesipox, Deerpox, Tanapox, Cotia virus and Volepox. For illustrative purposes, the encoded M2 protein orthologs of Horsepox, Variola virus, Monkeypox, Camelpox, cowpox display more than 90% identity with the reference Cop m2 protein (as represented by SEQ ID NO: 1) and those of myxoma, Skunk, Cotia and Volepox viruses shows respectively 50%, 74%, 70% and 72% sequence identity with the CopVV m2 protein, as illustrated in Table 1.

Table 1 provides an overview of the Genbank's accession numbers for the genomic sequences of various poxviruses comprising a M2L locus in the native context and an indication of the amino acid identity of their m2 protein with respect to Cop m2 protein (Uniprot's accession number P21092 and SEQ ID NO: 1).

| | Poxvirus | Genbank | % protein |
|---|---|---|---|
| Genus | name | reference | identity |
| orthopoxvirus | Vaccinia virus | AAA48004.1 | 100 |
| | Rabbitpox virus | AAS49736.1 | 100 |
| | Horsepox virus | ABH08137.1 | 99 |
| | Cowpox virus | ADZ29155.1 and | 99 and 92 |
| | | SNB53780.1 | |
| | Monkeypox virus | AAY97225.1 | 98 |
| | Variola major | AAA60767.1 | 97 |
| | Taterapox virus | ABD97599.1 | 97 |
| | Camelpox virus | AAL73736.1 | 96 |
| | Raccoonpox virus | AKJ93661.1 | 75 |
| | Skunkpox virus | AOP31509.1 | 74 |
| | Volepox virus | AOP31720.1 | 72 |
| Unclassified | Cotia virus | AFB76918.1 | 70 |
| Centapoxvirus | Yokapox virus | AEN03759.1 | 60 |
| | Murmansk poxvirus | AST09387.1 | 58 |
| Leporipoxvirus | Rabbit fibroma virus | AAF18030.1 | 50 |
| | Myxoma virus | AAF15042.1 | 50 |
| unclassified | Eptesipox virus | ASK51372.1 | 34 |
| Yatapoxvirus | Tanapox virus | ABQ43480.1 | 32 |
| | Yaba-like disease virus | CAC21247.1 | 29 |
| unclassified | Deerpoxvirus | ABI99004.1 | 28 |
| Cervidpoxvirus | (W-1170-84) | | |
| unclassified | Deerpoxvirus (White- | AUI80579.1 | 28 |
| | tailed deer pox) | | |

For sake of clarity, the gene nomenclature used herein to designate the poxviral M2L locus and the encoded m2 protein is that of vaccinia virus (and more specifically that of Copenhagen strain). It is also used herein for other poxviruses containing functionally equivalent M2L genes and m2 proteins to those referred herein unless otherwise indicated. Indeed, gene and respective gene product nomenclature may be different according to the poxvirus families, genus and strains but correspondences between vaccinia virus and other poxviruses are generally available in the literature. For illustrative purposes, equivalents of the VV M2L gene is designated M154L in myxoma's genome, CPXV040 or P2L in cowpox genome, 02L in Monkeypox genome, RPXV023 in rabbitpox genome and O2L or Q2L in variola virus genome.

However, the genome of a few poxviruses, such as the attenuated vaccinia virus MVA (Modified vaccinia virus Ankara) and the pseudocowpox virus (PCPV), in the native context, lacks a M2L locus (Antoine et al., 1998, Virology 244(2) 365-96) due to the large genomic deletions having occurred during the attenuation process. In the context of the invention, the term “poxvirus” does not include poxviruses which in the native context have genomic deletion(s) or mutation(s) encompassing M2L locus (or equivalent) which thus, lack a m2 polypeptide or encode a non-functional m2 protein such as Pseudocowpox virus (PCPV), MVA and NYVAC virus.

In one embodiment, the modified poxvirus of the present invention is generated or obtained from a Chordopoxvirinae, preferably selected from the group of genus consisting of Avipoxvirus, Capripoxvirus, Leporipoxvirus, Molluscipoxvirus, Orthopoxvirus, Parapoxvirus, Suipoxvirus, Cervidpoxvirus and Yatapoxvirus. Genomic sequences of these poxviruses are available in the art, notably in specialized databases such as Genbank or Refseq.

In a preferred embodiment, the modified poxvirus is generated or obtained from an Orthopoxvirus. Although any orthopox may be used, it is preferably selected from the group consisting of vaccinia virus (VV), cowpox (CPXV), raccoonpox (RCN), rabbitpox, Monkeypox, Horsepox, Volepox, Skunkpox, variola virus (or smallpox) and Camelpox. Particularly preferred is a vaccinia virus. Any vaccinia virus strain is appropriate in the context of the present invention (except MVA) including, without limitation, Western Reserve (WR), Copenhagen (Cop), Lister, LIVP, Wyeth, Tashkent, Tian Tan, Brighton, Ankara, LC16M8, LC16M0 strains, etc., with a specific preference for Lister, WR, Copenhagen and Wyeth strains. Genomic sequences thereof are available in the literature and Genbank (e.g. under accession numbers AY678276 (Lister), M35027 (Cop), AF095689 1 (Tian Tan) and AY243312.1 (WR). These viruses can also be obtained from virus collections (e.g. ATCC VR-1354 for WR, ATCC VR-1536 for Wyeth and ATCC VR-1549 for Lister).

In another embodiment, the modified poxvirus is generated or obtained from the Leporipoxvirus genus, with a preference for myxoma virus (which genomic sequences are disclosed in Genbank under accession number NP_051868.1). The M2L ortholog locus in myxoma virus is designated M154L locus and encodes a so-called gp120-like protein having the amino acid sequence shown in SEQ ID NO: 2 and displaying 50% identity with Cop-encoded m2 protein (SEQ ID NO: 1).

Defective m2 Function

As described above, the inability of a m2 protein to bind CD80 and/or CD86 co-stimulatory ligands may originate from a genetic lesion in the M2L locus or from an abnormal interaction impairing the m2 function either directly or indirectly. Specifically, a “defective m2 function” refers to a reduced capacity by at least 50%, at least 60%, at least 70%, at least 80%, at least 90%, at least 95%, or even total inability to bind CD80 (e.g. human) and CD86 (e.g. human) as compared to a native m2 protein (e.g. as found in supernatants of cells infected with a m2-positive poxvirus) as measured by a conventional assay such as competition ELISA assay.

A modified poxvirus may be engineered so as to be defective for m2 function by a number of ways known to those skilled in the art using conventional molecular techniques. In a preferred embodiment, the modified poxvirus comprises at least one genetic lesion in the native M2L locus that results in suppressed expression of the m2 protein by the virus. Such genetic lesion(s) include partial or total deletion and/or one or more non-silent mutation(s) (that translates in a change of amino acid residue(s)) either within the m2-coding sequence or in the regulatory elements controlling M2L expression. Said genetic lesion(s) preferably lead(s) to the synthesis of a defective m2 protein (unable to ensure the activity of the native protein as described above) or to the lack of m2 synthesis (no protein at all). For example, said genetic lesion is a partial or entire deletion of the M2L locus, e.g. a partial deletion extending from upstream the m2 coding sequences to at least 100 codons of the m2 coding sequence. Alternatively, or in combination, the M2L locus can be modified by point mutation (e.g. introduction of a STOP codon within the coding sequence), frameshift mutation (so as to modify the reading frame), insertional mutation (by insertion of one or more nucleotide(s) that disrupt the coding sequence) or by deletion or substitution of one or more residues involved in or responsible for the CD80 and/or CD86 binding function or any combination thereof. Also, a foreign nucleic acid can be introduced within the coding sequence to disrupt the m2 open reading frame. Also, the gene promoter can be deleted or mutated, thus inhibiting M2L expression. A person skilled in the art, based on the present disclosure would readily determine if a particular modification functionally inactivates m2, by comparing the wild-type and the mutated m2 protein for their ability to bind CD80 and/or CD86 as illustrated in the Example section.

Other Poxvirus Modifications

In one embodiment, the modified poxvirus of the present invention is further modified in a region other than M2L locus. Various additional modifications can be contemplated in the context of the invention.

One or more additional modification(s) encompassed by the present invention affect, for example, oncolytic activity (e.g. improved replication in dividing cells), safety (e.g. tumor selectivity), and/or virus-induced immunity compared to a poxvirus without such modifications. Exemplary modifications preferably concern viral genes involved in DNA metabolism, host virulence or IFN pathway (see e.g. Guse et al., 2011, Expert Opinion Biol. Ther. 11(5):595-608).

A particularly suitable gene to be disrupted is the thymidine kinase (tk)-encoding locus (J2R; Genbank accession number AAA48082). The tk enzyme is involved in the synthesis of deoxyribonucleotides. Tk is needed for viral replication in normal cells as these cells have generally low concentration of nucleotides whereas it is dispensable in dividing cells which contain high nucleotide concentration. Further, tk-defective viruses are known to have an increased selectivity to tumor cells. In one embodiment, the modified poxvirus is further modified in the J2R locus (preference for modification resulting in a suppressed expression of the viral tk protein), resulting in a modified poxvirus defective for both m2 and tk functions (m2-tk-poxvirus). Partial or complete deletion of said J2R locus as well as insertion of foreign nucleic acid in the J2R locus are contemplated in the context of the present invention to inactivate tk function. Such a modified m2-tk-poxvirus is desirably oncolytic.

Alternatively to or in combination with, the modified poxvirus may be further modified, in the 14L and/or F4L locus/loci (preference for modification leading to a suppressed expression of the viral ribonucleotide reductase (rr) protein), resulting in a modified poxvirus defective for both m2 and rr functions (m2 and rr-defective poxvirus). In the natural context, this enzyme catalyzes the reduction of ribonucleotides to deoxyribonucleotides that represents a crucial step in DNA biosynthesis. The viral enzyme is similar in subunit structure to the mammalian enzyme, being composed of two heterologous subunits, designed R1 and R2 encoded respectively by the 14L and F4L locus. Sequences for the 14L and F4L genes and their location in the genome of various poxvirus are available in public databases (see e.g. WO2009/065546). In the context of the invention, the poxvirus can be modified either in the 14L gene (encoding the r1 large subunit) or in the F4L gene (encoding the r2 small subunit) or both to provide a rr-defective poxvirus. e.g. by partial or complete deletion of said 14L and/or F4L locus/loci. Such a modified m2-rr-poxvirus is desirably oncolytic.

Also provided is a modified poxvirus further modified in the J2R and 14L/F4L loci (triple defective virus with modifications in the M2L, J2R and 14L loci; M2L, J2R and F4L loci or M2L, J2R, 14L and F4L loci), resulting in a modified poxvirus defective for m2, tk and rr activities (m2-, tk-rr-poxvirus). Such a modified tk-rr- and m2-poxvirus is desirably oncolytic.

In a preferred embodiment, such double and triple defective poxviruses preferably originate from an Orthopoxvirus, or a Leporipoxvirus as described above in connection with the m2-defective poxvirus. Particularly preferred is an oncolytic vaccinia virus other than MVA, with a specific preference for Lister, WR, Copenhagen, Wyeth strains. VV defective for tk and m2 activities and for tk, rr and m2 activities are particularly preferred, especially for use for stimulating or improving an immune response (e.g. a lymphocyte-mediated response against an antigen or epitope thereof) or for use for treating a proliferative disease as described herein

Other suitable additional modifications include those resulting in suppressed expression of one or more viral gene product(s) selected from the group consisting of the viral hemagglutinin (A56R); the serine protease inhibitor (B13R/B14R), the complement 4b binding protein (C3L), the VGF-encoding gene and the interferon modulating gene(s) (B8R or B18R). Another suitable modification comprises the inactivation of the F2L locus resulting in suppressed expression of the viral dUTPase (deoxyuridine triphosphatase) involved in both maintaining the fidelity of DNA replication and providing the precursor for the production of TMP by thymidylate synthase (WO2009/065547).

As for M2L, the gene nomenclature used herein is that of Cop VV strain. It is also used herein for the homologous genes of other poxviridae unless otherwise indicated and correspondence between Copenhagen and other poxviruses is available to the skilled person.

In still another embodiment, the modified poxvirus of the present invention is oncolytic. As used herein, the term “oncolytic” refers to the capacity of a poxvirus of selectively replicating in dividing cells (e.g. a proliferative cell such as a cancer cell) with the aim of slowing the growth and/or lysing said dividing cell, either in vitro or in vivo, while showing no or minimal replication in non-dividing (e.g. normal or healthy) cells. “Replication” (or any form of replication such as “replicate” and “replicating”, etc.,) means duplication of a virus that can occur at the level of nucleic acid or, preferably, at the level of infectious viral particle. The term “infectious” (or any form of infectious such as infect, infecting, etc.,) denotes the ability of a virus to infect and enter into a host cell or subject. Typically, an oncolytic poxvirus contains a viral genome packaged into a viral particle (encapsided and/or enveloped genome) although this term, in the context of the invention, may also encompass virus genome (e.g. genomic DNA) or part thereof.

Recombinant m2 Defective Poxvirus

In one embodiment, the modified poxviruses of the present invention is recombinant.

The term “recombinant” as used herein indicates that the poxvirus is engineered to express at least one foreign nucleic acid (also called recombinant gene, transgene or nucleic acid). In the context of the invention, the “foreign nucleic acid” that is inserted in the poxvirus genome is not found in or expressed by a naturally occurring poxvirus genome. Nevertheless, the foreign nucleic acid can be homologous or heterologous to the subject into which the recombinant poxvirus is introduced. More specifically, it can be of human origin or not (e.g. of bacterial, yeast or viral origin except poxviral). Advantageously, said recombinant nucleic acid encodes a polypeptide or is a nucleic acid sequence capable of binding at least partially (by hybridization) to a complementary cellular nucleic acid (e.g., DNA, RNA, miRNA) present in a diseased cell with the aim of inhibiting a gene involved in said disease. Such a recombinant nucleic acid may be a native gene or portion(s) thereof (e.g. cDNA), or any variant thereof obtained by mutation, deletion, substitution and/or addition of one or more nucleotides.

In one embodiment, the recombinant nucleic acid encodes a polypeptide which is of therapeutic or prophylactic interest (i.e. a polypeptide of therapeutic interest) when administered appropriately to a subject, leading to a beneficial effect on the course or a symptom of the pathological condition to be treated. A vast number of polypeptides of therapeutic interest may be envisaged. In a preferred embodiment, the modified poxvirus described herein is engineered to express at least one polypeptide selected from the group consisting of antigenic polypeptides (e.g. a tumor-associated or vaccinal antigen), polypeptides having nucleos/tide pool modulating function and immunomodulatory polypeptides. A recombinant modified poxvirus encoding a detectable gene product may also be useful in the context of the invention. “Engineered” as used herein refers to insertion of the one or more foreign nucleic acid in the viral genome at a suitable locus (e.g. in place of the J2R locus) under the control of appropriate regulatory elements to allow expression of said foreign nucleic acid in the host cell or organism.

Immunomodulatory Polypeptides

In one embodiment, the modified poxvirus described herein is engineered to express at least one immunomodulatory polypeptide. The term “immunomodulatory polypeptide” refers to a polypeptide targeting a component of a signalling pathway that can be involved in modulating an immune response either directly or indirectly. “Modulating” an immune response refers to any alteration in a cell of the immune system or in the activity of such a cell (e.g., a T cell). Such modulation includes stimulation or suppression of the immune system which can be manifested by an increase or decrease in the number of various cell types, an increase or decrease in the activity of these cells, or any other changes which can occur within the immune system. Preferably, such a polypeptide is capable of down-regulating at least partially an inhibitory pathway (antagonist) and/or of up-regulating at least partially a stimulatory pathway (agonist); in particular the immune pathway existing between an antigen presenting cell (APC) or a cancer cell and an effector T cell.

The immunomodulatory polypeptide for being expressed by the modified poxvirus described herein may act at any step of the T cell-mediated immunity including clonal selection of antigen-specific cells, T cell activation, proliferation, trafficking to sites of antigen and inflammation, execution of direct effector function and signaling through cytokines and membrane ligands. Each of these steps is regulated by counterbalancing stimulatory and inhibitory signals that in fine tune the response.

Suitable immunomodulatory polypeptides and methods of using them are described in the literature. Exemplary immunomodulatory polypeptides include, without limitation, cytokines, chemokines, ligands and antibodies or any combination thereof. The present invention encompasses a modified and preferably oncolytic, poxvirus encoding more than one immunomodulatory polypeptide (e.g. a cytokine and an antibody; a cytokine and a ligand; two cytokines; two immune checkpoint antibodies; a cytokine, a ligand and an antibody; an antibody and two cytokines; etc.).

In one embodiment, the immunomodulatory polypeptide to be expressed by the modified poxvirus described herein is a cytokine, preferably selected from the group consisting of IL-1, IL-2, IL-3, IL-4, IL-5, IL-6, IL-7, IL-8, IL-9, IL-10, IL-11, IL-12, IL-13, IL-14, IL-15, IL-16, IL-17, IL-18, IL-36, IFNa, IFNg and granulocyte macrophage colony stimulating factor (GM-CSF).

In another embodiment, the immunomodulatory polypeptide to be expressed by the modified poxvirus described herein is a chemokine, preferably selected from the group consisting of selected from the group comprising MIPIα, IL-8, CCL5, CCL17, CCL20, CCL22, CXCL9, CXCL10, CXCL11, CXCL13, CXCL12, CCL2, CCL19 and CCL21.

In still another embodiment, the immunomodulatory polypeptide to be expressed by the modified poxvirus described herein can be independently selected from the group consisting of peptides (e.g. peptide ligands), soluble domains of natural receptors and antibodies. Particularly appropriate in the context of the invention are antibodies that specifically bind an immune checkpoint protein, preferably selected from the group consisting of CD3, 4-1BB, GITR, OX40, CD27, CD40, PD1, PDL1, CTLA4, Tim-3, BTLA, Lag-3 and Tigit.

The term “specifically binds” refers to the capacity to a binding specificity and affinity for a particular target or epitope even in the presence of a heterogeneous population of other proteins and biologics. Thus, under designated assay conditions, the antibody binds preferentially to its target and does not bind in a significant amount to other components present in a test sample or subject. Preferably, such an antibody shows high affinity binding to its target with an equilibrium dissociation constant equal or below 1×10⁻⁶M (e.g. at least 0.5×10⁻⁶, 1×10⁻⁷, 1×10⁻⁸, 1×10⁹, 1×10⁻¹⁰, etc). Standard assays to evaluate the binding ability of an antibody to its target are known in the art, including for example, ELISAs, Western blots, RIAs and flow cytometry.

In the context of the invention, “antibody” (“Ab”) is used in the broadest sense and encompasses naturally occurring antibodies and those engineered by man; including synthetic, monoclonal, polyclonal antibodies as well as full length antibodies and fragments, variants or fusions thereof provided that such fragments, variants or fusions retain binding properties to the target protein. Such antibodies can be of any origin; human or non-human (e.g. rodent or camelid antibody) or chimeric. A nonhuman antibody can be humanized by recombinant methods to reduce its immunogenicity in man. The antibody may derive from any of the well-known isotypes (e.g. IgA, IgG and IgM) and any subclasses of IgG (IgG1, IgG2, IgG3, IgG4). In addition, it may be glycosylated, partially glycosylated or non-glycosylated. Unless the context indicates otherwise, the term “antibody” also includes an antigen-binding fragment of any of the aforementioned antibodies and includes a monovalent and a divalent fragment and single chain antibodies. The term antibody also includes multi-specific (e.g. bispecific) antibody so long as it exhibits the same binding specificity as the parental antibody. It is within the skill of the artisan to screen for the binding properties of a candidate antibody.

For illustrative purposes, full length antibodies are glycoproteins comprising at least two heavy (H) chains and two light (L) chains inter-connected by disulfide bonds. Each heavy chain comprises a heavy chain variable region (VH) and a heavy chain constant region which is made of three CH1, CH2 and CH3 domains (eventually with a hinge between CH1 and CH2). Each light chain comprises a light chain variable region (VL) and a light chain constant region which comprises one CL domain. The VH and VL regions comprise three hypervariable regions, named complementarity determining regions (CDR), interspersed with four conserved regions named framework regions (FR) in the following order: FR1-CDR1-FR2-CDR2-FR3-CDR3-FR4. The CDR regions of the heavy and light chains are determinant for the binding specificity. As used herein, a “humanized antibody” refers to a non-human (e.g. murine, camel, rat, etc) antibody whose protein sequence has been modified to increase its similarity to a human antibody (i.e. produced naturally in humans). The process of humanization is well known in the art and typically is carried out by substituting one or more residue of the FR regions to look like human immunoglobulin sequence whereas the vast majority of the residues of the variable regions (especially the CDRs) are not modified and correspond to those of a non-human immunoglobulin. A “chimeric antibody” comprises one or more element(s) of one species and one or more element(s) of another species, for example, a non-human antibody comprising at least a portion of a constant region (Fc) of a human immunoglobulin.

Representative examples of antigen-binding fragments are known in the art, including Fab, Fab′, F(ab′)2, dAb, Fd, Fv, scFv, ds-scFv and diabody. A particularly useful antibody fragment is a single chain antibody (scFv) comprising the two domains of a Fv fragment, VL and VH, that are fused together, eventually with a linker to make a single protein chain.

In one embodiment, the antibody to be expressed by the modified poxvirus described herein is a monoclonal antibody or a single chain antibody that specifically binds to a molecule at the surface of a T cell and preferably to an immunosuppressive receptor involved in the regulation of T cell activation. Further to binding ability, such an antibody is further capable of inhibiting the biological activity of said immunosuppressive receptor.

Particularly preferred embodiments are directed to a modified and preferably oncolytic poxvirus expressing an antagonist antibody that specifically binds to PD-L1 or CTLA4 and preferably inhibits the biological activity of such a receptor, notably by inhibiting interaction with the co-stimulatory CD80 and/or CD86 ligand(s).

In a preferred embodiment, the antagonist antibody to be expressed by the recombinant modified poxvirus described herein is an anti-CTLA-4 antibody that specifically binds to a mammalian CTLA-4 (e.g. human CTLA-4) and inhibits its capacity to deliver an immunosuppressive signal (e.g. by blocking the binding of CTLA-4 to the CD80 and CD86 ligands).

With a conventional poxvirus carrying the M2L locus in its genome, the expressed anti-CTLA-4 antibody will act to inhibit the CTLA-4-mediated immunosuppressive signal but the in situ produced M2 protein will interact with CD80 and CD86 ligands, thus reducing or inhibiting the CD28-mediated co-stimulatory signal. In contrast, the anti-CTLA-4-expressing modified (i.e. m2-defective) poxvirus described herein which lacks m2 function will be able to both inhibit the CTLA-4-mediated immunosuppressive signal and to redirect the immune response towards CD28-mediated co-stimulatory signals.

A number of anti CTLA-4 antibodies are available in the art (see e.g. those described in U.S. Pat. No. 8,491,895, WO2000/037504, WO2007/113648, WO2012/122444 and WO2016/196237 among others) and a fistful of them have been FDA approved for the last decade or are under advanced clinical development. Representative examples of anti-CTLA-4 antibodies usable in the present disclosure are, e.g., ipilimumab marketed by Bristol Myer Squibb as Yervoy® (see e.g. U.S. Pat. Nos. 6,984,720; 8,017,114), MK-1308 (Merck), AGEN-1884 (Agenus Inc.; WO2016/196237) and tremelimumab (AstraZeneca; U.S. Pat. Nos. 7,109,003 and 8,143,379) and single chain anti-CTLA4 antibodies (see e.g. WO97/20574 and WO2007/123737).

Preferred embodiments are directed to (i) a modified (and preferably oncolytic) poxvirus with a preference for an oncolytic vaccinia virus defective for both m2 and tk function (resulting from inactivating mutations in both the M2L and the J2R loci) encoding an anti-CTLA-4 antibody; (ii) a modified (and preferably oncolytic) poxvirus with a preference for an oncolytic vaccinia virus, defective for m2 and rr activities (resulting from inactivating mutations in both the M2L locus and the 14L and/or F4L gene(s)) encoding an anti-CTLA-4 antibody and (iii) a modified (and preferably oncolytic) poxvirus with a preference for an oncolytic vaccinia virus, defective for m2, tk and rr activities (resulting from inactivating mutations in the M2L, J2R and 14L/F4L loci) encoding an anti-CTLA-4 antibody.

In certain embodiments, the anti-CTLA-4 antibody is ipilimumab.

In certain embodiments, the anti-CTLA-4 antibody is tremelimumab.

Another preferred example of immunomodulatory polypeptides suitable for expression by the modified poxvirus described herein is represented by an antibody specifically binding PDL-1 (programmed Death Ligand-1) and inhibiting its biological activity. Formation of a PD-1/PD-L1 receptor/ligand complex leads to inhibition of CD8+ T cells, and therefore inhibition of an immune response. PD-L1 is one of two cell surface glycoprotein ligands for PD-1 (the other being PD-L2) that downregulates T cell activation and cytokine secretion upon binding to PD-1. The complete human PD-L1 sequence can be found under GenBank Accession No. Q9NZQ7.

Antagonist anti PD-L1 antibodies are available in the art from various providers such as Merck, sigma Aldrich and Abcam and some have been FDA approved or under advanced late clinical development. Representative examples of anti PD-L1 antibodies usable in the present disclosure are e.g., BMS-936559 (under development by Bristol Myer Squibb also known as MDX-1105; WO2013/173223), atezolizumab (under development by Roche; also known as TECENTRIQ®; U.S. Pat. No. 8,217,149), durvalumab (AstraZeneca; also known as EVIFINZI™; WO2011/066389), MPDL3280A (under development by Genentech/Roche), as well avelumab (developed by Merck and Pfizer under trade name Bavencio; WO2013/079174), STI-1014 (Sorrento; WO2013/181634) and CX-072 (Cytomx; WO2016/149201). The corresponding nucleotide sequences can be cloned or isolated according to standard techniques based on the information disclosed in the available literature.

Preferred embodiments are directed to (i) a modified (and preferably oncolytic) poxvirus with a preference for an oncolytic vaccinia virus defective for both m2 and tk function (resulting from inactivating mutations in both the M2L and the J2R loci) encoding an anti-PD-L1 antibody; (ii) a modified (and preferably oncolytic) poxvirus with a preference for an oncolytic vaccinia virus, defective for m2 and rr activities (resulting from inactivating mutations in both the M2L locus and the 14L and/or F4L gene(s)) encoding an anti-PD-L1 antibody and (iii) a modified (and preferably oncolytic) poxvirus with a preference for an oncolytic vaccinia virus, defective for m2, tk and rr activities (resulting from inactivating mutations in the M2L, J2R and 14L/F4L loci) encoding an anti-PD-L1 antibody.

In certain embodiments, the anti-PD-L1 antibody is atezolizumab.

In certain embodiments, the anti-PD-L1 antibody is durvalumab.

In certain embodiments, the anti-PD-L1 antibody is avelumab.

Other embodiments are directed to (i) a modified and preferably oncolytic poxvirus with a preference for an oncolytic vaccinia virus defective for both m2 and tk function (resulting from inactivating mutations in both the M2L and the J2R loci) encoding an anti-CTLA-4 antibody and an anti-PD-L1 antibody; (ii) a modified and preferably oncolytic poxvirus with a preference for an oncolytic vaccinia virus, defective for m2 and rr activities (resulting from inactivating mutations in both the M2L locus and the 14L and/or F4L gene(s)) encoding an anti-CTLA-4 antibody and an anti-PD-L1 antibody and (iii) a modified and preferably oncolytic poxvirus with a preference for an oncolytic vaccinia virus, defective for m2, tk and rr activities (resulting from inactivating mutations in the M2L, J2R and 14L/F4L loci) encoding an anti-CTLA-4 antibody and an anti-PD-L1 antibody.

In certain embodiments, the anti-CTLA-4 antibody is ipilimumab and the anti-PD-L1 antibody is avelumab.

Antigenic Polypeptides

The term “antigenic” refers to the ability to induce or stimulate a measurable immune response in a subject into which the recombinant poxvirus described herein encoding the polypeptide qualified as antigenic has been introduced. The stimulated or induced immune response against the antigenic polypeptide expressed by said recombinant poxvirus can be humoral and/or cellular (e.g. production of antibodies, cytokines and/or chemokines involved in the activation of effector immune cells). The stimulated or induced immune response usually contributes in a protective effect in the administered subject. A vast variety of direct or indirect biological assays are available in the art to evaluate the antigenic nature of a polypeptide either in vivo (animal or human subjects), or in vitro (e.g. in a biological sample). For example, the ability of a particular antigen to stimulate innate immunity can be performed by for example measurement of the NK/NKT-cells (e.g. representativity and level of activation), as well as, IFN-related cytokine and/or chemokine producing cascades, activation of TLRs (for Toll-like receptor) and other markers of innate immunity (Scott-Algara et al., 2010 PLOS One 5(1), e8761; Zhou et al., 2006, Blood 107, 2461-2469; Chan, 2008, Eur. J. Immunol. 38, 2964-2968). The ability of a particular antigen to stimulate a cell-mediated immune response can be performed for example by quantification of cytokine(s) produced by activated T cells including those derived from CD4+ and CD8+ T-cells using routine bioassays (e.g. characterization and/or quantification of T cells by ELISpot, by multiparameters flow cytometry, ICS (for intracellular cytokine staining), by cytokine profile analysis using multiplex technologies or ELISA), by determination of the proliferative capacity of T cells (e.g. T cell proliferation assays by [³H] thymidine incorporation assay), by assaying cytotoxic capacity for antigen-specific T lymphocytes in a sensitized subject or by identifying lymphocyte subpopulations by flow cytometry and by immunization of appropriate animal models, as described herein.

It is contemplated that the term antigenic polypeptide encompasses native antigen as well as fragment (e.g. epitopes, immunogenic domains, etc) and variant thereof, provided that such fragment or variant is capable of being the target of an immune response. Preferred antigenic polypeptides for use herein are tumor-associated antigens. It is within the scope of the skilled artisan to select the one or more antigenic polypeptide that is appropriate for treating a particular pathological condition.

In one embodiment, the antigenic polypeptide(s) encoded by the recombinant modified poxvirus is/are cancer antigen(s) (also called tumor-associated antigens or TAA) that is associated with and/or serve as markers for cancers. Cancer antigens encompass various categories of polypeptides, e.g. those which are normally silent (i.e. not expressed) in healthy cells, those that are expressed only at low levels or at certain stages of differentiation and those that are temporally expressed such as embryonic and foetal antigens as well as those resulting from mutation of cellular genes, such as oncogenes (e.g. activated ras oncogene), proto-oncogenes (e.g. ErbB family), or proteins resulting from chromosomal translocations.

Numerous tumor-associated antigens are known in the art. Exemplary tumor antigens include without limitation, colorectal associated antigen (CRC), Carcinoembryonic Antigen (CEA), Prostate Specific Antigen (PSA), BAGE, GAGE or MAGE antigen family, p53, mucin antigens (e.g. MUC1), HER2/neu, p21ras, hTERT, Hsp70, iNOS, tyrosine kinase, mesothelin, c-erbB-2, alpha fetoprotein, AM-1, among many others, and any immunogenic epitope or variant thereof.

The tumor-associated antigens may also encompass neo-epitopes/antigens that have emerged during the carcinogenesis process in a cancer cell and comprising one or more mutation(s) of amino acid residue(s) with respect to a corresponding wild-type antigen. Typically, it is found in cancer cells or tissues obtained from a patient but not found in a sample of normal cells or tissues obtained from a patient or a heathy individual.

The tumor-associated antigens may also encompass antigens encoded by pathogenic organisms that are capable of inducing a malignant condition in a subject (especially chronically infected subject) such as RNA and DNA tumor viruses (e.g. human papillomavirus (HPV), hepatitis C virus (HCV), hepatitis B virus (HBV), Epstein Barr virus (EBV), etc) and bacteria (e.g. Helicobacter pilori).

In another embodiment, the antigenic polypeptide(s) encoded by the recombinant modified poxvirus is/are vaccinal antigen(s) that, when delivered to a human or animals subject, aim(s) at protecting therapeutically or prophylactically against infectious diseases. Numerous vaccine antigens are known in the art. Exemplary vaccine antigens include but are not limited to cellular antigens, viral, bacterial or parasitic antigens. Cellular antigens include the mucin 1 (MUC1) glycoprotein. Viral antigens include for example antigens from hepatitis viruses A, B, C, D and E, immunodeficiency viruses (e.g. HIV), herpes viruses, cytomegalovirus, varicella zoster, papilloma viruses, Epstein Barr virus, influenza viruses, para-influenza viruses, coxsakie viruses, picorna viruses, rotaviruses, respiratory syncytial viruses, rhinoviruses, rubella virus, papovirus, mumps virus, measles virus and rabbies virus. Some non-limiting examples of HIV antigens include gp120 gp40, gp160, p24, gag, pol, env, vif, vpr, vpu, tat, rev, nef tat, nef. Some non-limiting examples of human herpes virus antigens include gH, gL gM gB gC gK gE or gD or Immediate Early protein such asICP27, ICP47, ICP4, ICP36 from HSV1 or HSV2. Some non-limiting examples of cytomegalovirus antigens include gB. Some non-limiting examples of derived from Epstein Barr virus (EBV) include gp350. Some non-limiting examples of Varicella Zoster Virus antigens include gp1, 11, 111 and IE63. Some non-limiting examples of hepatitis C virus antigens includes env E1 or E2 protein, core protein, NS2, NS3, NS4a, NS4b, NS5a, NS5b, p7. Some non-limiting examples of human papilloma viruses (HPV) antigens include L1, L2, E1, E2, E3, E4, E5, E6, E7. Antigens derived from other viral pathogens, such as Respiratory Syncytial virus (e.g. F and G proteins), parainfluenza virus, measles virus, mumps virus, flaviviruses (e.g. Yellow Fever Virus, Dengue Virus, Tick-borne encephalitis virus, Japanese Encephalitis Virus) and Influenza virus cells (e.g. HA, NP, NA, or M proteins) can also be used in accordance with the present invention. Bacterial antigens include for example antigens from Mycobacteria causing TB, leprosy, pneumocci, aerobic gram negative bacilli, mycoplasma, staphyloccocus, streptococcus, salmonellae, chlamydiae, neisseriae and the like. Parasitic antigenic polypeptides include for example antigens from malaria, leishmaniasis, trypanosomiasis, toxoplasmosis, schistosomiasis and filariasis.

Nucleoside Pool Modulators

In one embodiment, the modified poxvirus described herein carries in its genome one or more recombinant gene(s) having nucleoside pool modulator function. Representative examples include without limitation cytidine deaminase and notably yeast cytidine deaminase (CDD1) or human cytidine deaminase (hCD) (see WO2018/122088); polypeptides acting on metabolic and immune pathways (e.g., adenosine deaminase and notably the human adenosine deaminase huADA1 or huADA2; see EP17306012.0); polypeptides acting on the apoptotic pathway; endonucleases (like restriction enzymes, CRISPR/Cas9), and target-specific RNAs (e.g., miRNA, shRNA, siRNA).

Detectable Gene Products

Typically, such a polypeptide is detectable by spectroscopic, photochemical, biochemical, immunochemical, chemical, or other physical means and thus may permit to identify the recombinant poxvirus within a host cell or subject. Non-limiting examples of suitable detectable gene products includes mCherry, Emerald, firefly luciferase and green fluorescent proteins (GFP and enhanced version thereof e-GFP) detectable by fluorescent means as well as beta-galactosidase detectable by colorimetric means.

Expression of the Recombinant Gene

The nucleotide sequence encoding the polypeptide of therapeutic interest such as those cited above may be easily obtained by standard molecular biology techniques (e.g. PCR amplification, cDNA cloning, chemical synthesis) using sequence data accessible in the art and the information provided herein. For example, methods for cloning antibodies, fragments and analogs thereof are known in the art (see e.g. Harlow and Lane, 1988, Antibodies—A laboratory manual; Cold Spring Harbor Laboratory, Cold Spring Harbor N.Y.). Antibody-encoding nucleic acid molecule may be isolated from the producing hybridoma (e.g. Cole et al. in Monoclonal antibodies and Cancer Therapy; Alan Liss pp 77-96), immunoglobulin gene libraries, or from any available source or the nucleotide sequence may be generated by chemical synthesis.

In addition, the recombinant nucleic acid can be optimized for providing high level expression in a particular host cell or subject. It has been indeed observed that, the codon usage patterns of organisms are highly non-random and the use of codons may be markedly different between different hosts. For example, the therapeutic gene may be from bacterial, viral or lower eukaryote origin and thus have an inappropriate codon usage pattern for efficient expression in higher eukaryotic cells (e.g. human). Typically, codon optimization is performed by replacing one or more “native” (e.g. bacterial, viral or yeast) codon corresponding to a codon infrequently used in the host organism by one or more codon encoding the same amino acid which is more frequently used. It is not necessary to replace all native codons corresponding to infrequently used codons since increased expression can be achieved even with partial replacement.

Further to optimization of the codon usage, expression in the host cell or subject can further be improved through additional modifications of the recombinant nucleic sequence(s). For example, various modifications may be envisaged so as to prevent clustering of rare, non-optimal codons being present in concentrated areas and/or to suppress or modify “negative” sequence elements which are expected to negatively influence expression levels. Such negative sequence elements include without limitation the regions having very high (>80%) or very low (<30%) GC content; AT-rich or GC-rich sequence stretches; unstable direct or inverted repeat sequences; R A secondary structures; and/or internal cryptic regulatory elements such as internal TATA-boxes, chi-sites, ribosome entry sites, and/or splicing donor/acceptor sites.

In accordance with the present invention, each of the one or more recombinant nucleic acid molecule(s) is operably linked to suitable regulatory elements for its expression in a host cell or subject. As used herein, the term “regulatory elements” or “regulatory sequence” refers to any element that allows, contributes or modulates the expression of the encoding nucleic acid (s) in a given host cell or subject, including replication, duplication, transcription, splicing, translation, stability and/or transport of the nucleic acid(s) or its derivative (i.e. m RNA). As used herein, “operably linked” means that the elements being linked are arranged so that they function in concert for their intended purposes. For example, a promoter is operably linked to a nucleic acid molecule if the promoter effects transcription from the transcription initiation to the terminator of said nucleic acid molecule in a permissive host cell.

It will be appreciated by those skilled in the art that the choice of the regulatory sequences can depend on such factors as the nucleic acid itself, the virus into which it is inserted, the host cell or subject, the level of expression desired, etc. The promoter is of special importance. In the context of the invention, it can be constitutive directing expression of the nucleic acid molecule in many types of host cells or specific to certain host cells (e.g. liver-specific regulatory sequences) or regulated in response to specific events or exogenous factors (e.g. by temperature, nutrient additive, hormone, etc.,) or according to the phase of a viral cycle (e.g. late or early). One may also use promoters that are repressed during the production step in response to specific events or exogenous factors, in order to optimize virus production and circumvent potential toxicity of the expressed polypeptide(s).

Poxvirus promoters are particularly adapted for expression of the recombinant gene by the modified poxvirus described herein. Representative examples include without limitation the vaccinia 7.5K, H5R, 11K7.5 (Erbs et al., 2008, Cancer Gene Ther. 15(1): 18-28), TK, p28, p11, pB2R, pA35R and K1L promoters, as well as synthetic promoters such as those described in Chakrabarti et al. (1997, Biotechniques 23: 1094-7; Hammond et al, 1997, J. Virol Methods 66: 135-8; and Kumar and Boyle, 1990, Virology 179: 151-8) as well as early/late chimeric promoters.

Those skilled in the art will appreciate that the regulatory elements controlling the expression of the nucleic acid molecule(s) inserted into the poxviral genome may further comprise additional elements for proper initiation, regulation and/or termination of transcription (e.g. polyA transcription termination sequences), mRNA transport (e.g. nuclear localization signal sequences), processing (e.g. splicing signals), and stability (e.g. introns and non-coding 5′ and 3′ sequences), translation (e.g. an initiator Met, tripartite leader sequences, IRES ribosome binding sites, signal peptides, etc.).

When appropriate, it may be advantageous that the recombinant polypeptide include additional regulatory elements to facilitate its expression, trafficking and biological activity. For example, a signal peptide may be included for facilitating secretion from the infected cell. The signal peptide is typically inserted at the N-terminus of the protein immediately after the Met initiator. The choice of signal peptides is wide and is accessible to persons skilled in the art. One may also envisage addition of a trans-membrane domain to facilitate anchorage of the encoded polypeptide(s) in a suitable membrane (e.g. the plasmic membrane) of the infected cells. The trans-membrane domain is typically inserted at the C-terminus of the protein just before or at close proximity of the STOP codon. A vast variety of trans-membrane domains are available in the art (see e.g. WO99/03885).

As an additional example, a peptide tag (typically a short peptide sequence able to be recognized by available antisera or compounds) may be also be added for following expression, trafficking or purification of the encoded gene product. A vast variety of tag peptides can be used in the context of the invention including, without limitation, PK tag, FLAG octapeptide, MYC tag, HIS tag (usually a stretch of 4 to 10 histidine residues) and e-tag (U.S. Pat. No. 6,686,152). The tag peptide(s) may be independently positioned at the N-terminus of the protein or alternatively at its C-terminus or alternatively internally or at any of these positions when several tags are employed. Tag peptides can be detected by immunodetection assays using anti-tag antibodies.

As another example, the glycosylation can be altered so as to increase biological activity of the encoded gene product. Such modifications can be accomplished, for example, by mutating one or more residues within the site(s) of glycosylation. Altered glycosylation patterns may increase the ADCC ability of antibodies and/or their affinity for their target.

Another approach that may be pursued in the context of the present invention is coupling of the recombinant gene product encoded by the modified poxvirus described herein to an external agent such as a cytotoxic agent and/or a labelling agent. As used herein, the term “cytotoxic agent” refers to a compound that is directly toxic to cells (e.g. preventing their reproduction or growth) such as toxins (e. g. an enzymatically active toxin of bacterial, fungal, plant or animal origin, or fragments thereof). As used herein, “a labeling agent” refers to a detectable compound. The labeling agent may be detectable by itself (e. g., radioactive isotope labels or fluorescent labels) or, in the case of an enzymatic label, may catalyze chemical modification of a substrate compound which is detectable. The coupling may be performed by genetic fusion between the therapeutic polypeptide and the external agent.

Insertion of the recombinant nucleic acid(s) (equipped with appropriate regulatory elements) in the poxvirus genome is made by conventional means, either using appropriate restriction enzymes or, preferably by homologous recombination.

In one further aspect, the present invention provides a method for generating the modified poxvirus described herein, and particularly a recombinant and oncolytic poxvirus, by homologous recombination between a transfer plasmid comprising the recombinant nucleic acid (with its regulatory elements) flanked in 5′ and 3′ with viral sequences respectively present upstream and downstream the insertion site and a virus genome. In one embodiment, said method comprise a step of generating said transfer plasmid (e.g. by conventional molecular biology methods) and a step of introducing said transfer plasmid into a suitable host cell, notably together with a poxvirus genome (e.g. a M2L-inactivated virus) comprising the flanking sequence present in the transfer plasmid. Preferably, the transfer plasmid is introduced into the host cell by transfection and the virus by infection.

The size of each flanking viral sequence may vary. It is usually at least 100 bp and at most 1500 bp, with a preference for approximately 150 to 800 bp on each side of the recombinant nucleic acid, advantageously from 180 to 600 bp, preferably from 200 to 550 bp and more preferably from 250 to 500 bp.

The recombinant nucleic acid molecule(s) can independently be inserted at any location of the poxviral genome and insertion can be performed by routine molecular biology well known in the art. Various sites of insertion may be considered, e.g. in a non-essential viral gene, in an intergenic region, or in a non-coding portion of the poxvirus genome. J2R locus is particularly appropriate in the context of the invention. As described above, upon insertion of the foreign nucleic acid(s) into the poxvirus genome, the viral locus at the insertion site may be deleted at least partially, e.g. resulting in suppressed expression of the viral gene product encoded by the entirely or partially deleted locus and a defective virus for said virus function.

In certain embodiments, identification of the modified poxvirus may be facilitated by the use of a selection and/or a detectable gene. In preferred embodiments, the transfer plasmid further comprises a selection marker with a specific preference for the GPT gene (encoding a guanine phosphoribosyl transferase) permitting growth in a selective medium (e.g. in the presence of mycophenolic acid, xanthine and hypoxanthine) or a detectable gene encoding a detectable gene product such as GFP, e-GFP or mCherry. In addition, the use of an endonuclease capable of providing a double-stranded break in said selection or detectable gene may also be considered. Said endonuclease may be in the form of a protein or expressed by an expression vector.

Homologous recombination permitting to generate the modified poxvirus is preferably carried out in appropriate host cells (e.g. HeLa or CEF cells).

Production of the Poxvirus

Typically, the modified poxvirus of the invention is produced into a suitable host cell line using conventional techniques including culturing the transfected or infected host cell under suitable conditions so as to allow the production and recovery of infectious poxviral particles.

Therefore, in another aspect, the present invention relates to a method for producing the modified poxvirus described herein. Preferably said method comprises the steps of a) preparing a producer cell line, b) transfecting or infecting the prepared producer cell line with the modified poxvirus, c) culturing the transfected or infected producer cell line under suitable conditions so as to allow the production of the virus (e.g. infectious poxviral particles), d) recovering the produced virus from the culture of said producer cell line and optionally e) purifying said recovered virus.

In one embodiment, the producer cell is a mammalian (e.g. human or non-human) cell selected from the group consisting of HeLa cells (e.g. ATCC-CRM-CCL-2™ or ATCC-CCL-2.2™), HER96, PER-C6 (Fallaux et al., 1998, Human Gene Ther. 9: 1909-17) and hamster cell lines such as BHK-21 (ATCC CCL-10) or an avian cell such as one of those described in WO2005/042728, WO2006/108846, WO2008/129058, WO2010/130756, WO2012/001075, etc) as well as a primary chicken embryo fibroblast (CEF) prepared from chicken embryos obtained from fertilized eggs.

Producer cells are preferably cultured in an appropriate medium which can, if needed, be supplemented with serum and/or suitable growth factor(s) or not (e.g. a chemically defined medium preferably free from animal- or human-derived products). An appropriate medium may be easily selected by those skilled in the art depending on the producer cells. Such media are commercially available. Producer cells are preferably cultivated at a temperature comprised between +30° C. and +38° C. (more preferably at approximately 37° C.) for between 1 and 8 days before infection. If needed, several passages of 1 to 8 days may be made in order to increase the total number of cells.

In step b), producer cells are infected by the modified poxvirus under appropriate conditions using an appropriate multiplicity of infection (MOI) to permit productive infection of producer cells. For illustrative purposes, an appropriate MOI ranges from 10⁻³ to 20, with a specific preference for a MOI comprises from 0.01 to 5 and more preferably 0.03 to 1. Infection step is carried out in a medium which may be the same as or different from the medium used for the culture of producer cells.

In step c), infected producer cells are then cultured under appropriate conditions well known to those skilled in the art until progeny poxvirus (e.g. infectious virus particles) is produced. Culture of infected producer cells is also preferably performed in a medium which may be the same as or different from the medium/media used for culture of producer cells and/or for infection step, at a temperature between +32° C. and +37° C., for 1 to 5 days.

In step d), the poxvirus produced in step c) is collected from the culture supernatant and/or the producer cells. Recovery from producer cells may require a step allowing the disruption of the producer cell membrane to allow the liberation of the virus. The disruption of the producer cell membrane can be induced by various techniques well known to those skilled in the art, including but not limited to freeze/thaw, hypotonic lysis, sonication, microfluidization, high shear (also called high speed) homogenization or high-pressure homogenization.

The recovered poxvirus may then be at least partially purified before being distributed in doses and used according to the present invention. A vast number of purification steps and methods is available in the art, including e.g. clarification, enzymatic treatment (e.g. endonuclease, protease, etc), chromatographic and filtration steps. Appropriate methods are described in the art (see e.g. WO2007/147528; WO2008/138533, WO2009/100521, WO2010/130753, WO2013/022764).

In one embodiment, the present invention also provides a cell infected with the modified poxvirus described herein.

Composition

The present invention also provides a composition comprising a therapeutically effective amount of the modified poxvirus described herein (active agent) and a pharmaceutically acceptable vehicle. Such a composition may be administered once or several times and via the same or different routes

A “therapeutically effective amount” corresponds to the amount of modified poxvirus that is sufficient for producing one or more beneficial results. Such a therapeutically effective amount may vary as a function of various parameters, in particular the mode of administration; the disease state; the age and weight of the subject; the ability of the subject to respond to the treatment; kind of concurrent treatment; the frequency of treatment; and/or the need for prevention or therapy. When prophylactic use is concerned, the composition of the invention is administered at a dose sufficient to prevent or to delay the onset and/or establishment and/or relapse of the proliferative disease (such as cancer), especially in a subject at risk. For “therapeutic” use, the composition is administered to a subject diagnosed as having a proliferative disease (such as cancer) with the goal of treating the disease, eventually in association with one or more conventional therapeutic modalities. In particular, a therapeutically effective amount could be that amount necessary to cause an observable improvement of the clinical status over the baseline status or over the expected status if not treated, e.g. reduction in the tumor number; reduction in the tumor size, reduction in the number or extend of metastasis, increase in the length of remission, stabilization (i.e. not worsening) of the state of disease, delay or slowing of disease progression or severity, amelioration or palliation of the disease state, prolonged survival, better response to the standard treatment, improvement of quality of life, reduced mortality, etc. For example, techniques routinely used in laboratories (e.g. flow cytometry, histology, medical imaging) may be used to perform tumor surveillance.

A therapeutically effective amount could also be the amount necessary to cause the development of an effective non-specific (innate) and/or specific (adaptative) immune response. Typically, development of an immune response, in particular T cell response, can be evaluated in vitro, in suitable animal models or using biological samples collected from the subject (ELISA, flow cytometry, histology, etc). One may also use various available antibodies so as to identify different immune cell populations involved in anti-tumor response that are present in the treated subjects, such as cytotoxic T cells, activated cytotoxic T cells, natural killer cells and activated natural killer cells. An improvement of the clinical status can be easily assessed by any relevant clinical measurement typically used by physicians or other skilled healthcare staff.

The term “pharmaceutically acceptable vehicle” is intended to include any and all carriers, solvents, diluents, excipients, adjuvants, dispersion media, coatings, antibacterial and antifungal agents, absorption agents and the like compatible with administration in mammals and in particular human subjects. Non-limiting examples of pharmaceutically acceptable vehicles include water, NaCl, normal saline solutions, lactated Ringer's, saccharide solution (e.g. glucose, trehalose, saccharose, dextrose, etc) alcohols, oils, gelatins, carbohydrates such as lactose, amylose or starch, fatty acid esters, hydroxymethycellulose, and the like as well as other aqueous physiologically balanced salt solutions may be used (see for example the most current edition of Remington: The Science and Practice of Pharmacy, A. Gennaro, Lippincott, Williams&Wilkins).

In one embodiment, the composition is formulated appropriately to ensure the stability of the modified poxvirus active agent under the conditions of manufacture and long-term storage (i.e. for at least 6 months, with a preference for at least two years) at freezing (e.g. between −70° C. and −10° C.), refrigerated (e.g. 4° C.) or ambient (e.g. 20-25° C.) temperature. Such formulations generally include a liquid carrier such as aqueous solutions.

Advantageously, the composition is suitably buffered for human use, preferably at physiological or slightly basic pH (e.g. from approximately pH 7 to approximately pH 9 with a specific preference for a pH comprised between 7 and 8 and more particularly close to 7.5). Suitable buffers include without limitation TRIS (tris(hydroxymethyl)methylamine), TRIS-HCl (tris(hydroxymethyl)methylamine-HCl), HEPES (4-2-hydroxyethyl-1-piperazineethanesulfonic acid), phosphate buffer (e.g. PBS), ACES (N-(2-Acetamido)-aminoethanesulfonic acid), PIPES (Piperazine-N,N′-bis(2-ethanesulfonic acid)), MOPSO (3-(N-Morpholino)-2-hydroxypropanesulfonic acid), MOPS (3-(N-morpholino)propanesulfonic acid), TES (2-{[tris(hydroxymethyl)methyl]amino}ethanesulfonic acid), DIPSO (3-[bis(2-hydroxyethyl)amino]-2-hydroxypropane-1-sulfonic acid), MOBS (4-(N-morpholino)butanesulfonic acid), TAPSO (3-[N-Tris(hydroxymethyl)methylamino]-2-hydroxypropanesulfonic Acid), HEPPSO (4-(2-Hydroxyethyl)-piperazine-1-(2-hydroxy)-propanesulfonic acid), POPSO (2-hydroxy-3-[4-(2-hydroxy-3-sulfopropyl)piperazin-1-yl]propane-1-sulfonic acid), TEA (triethanolamine), EPPS (N-(2-Hydroxyethyl)-piperazine-N′-3-propanesulfonic acid), and TRICINE (N-[Tris(hydroxymethyl)-methyl]-glycine). Preferably, said buffer is selected from TRIS-HCl, TRIS, Tricine, HEPES and phosphate buffer comprising a mixture of Na₂HPO₄ and KH₂PO₄ or a mixture of Na₂HPO₄ and NaH₂PO₄. Said buffer (in particular those mentioned above and notably TRIS-HCl) is preferably present in a concentration of 10 to 50 mM.

It might be beneficial to also include in the formulation a monovalent salt so as to ensure an appropriate osmotic pressure. Said monovalent salt may notably be selected from NaCl and KCl, preferably said monovalent salt is NaCl, preferably in a concentration of 10 to 500 mM.

The composition may also be formulated so as to include a cryoprotectant for protecting the modified poxvirus at low storage temperature. Suitable cryoprotectants include without limitation sucrose (or saccharose), trehalose, maltose, lactose, mannitol, sorbitol and glycerol, preferably in a concentration of 0.5 to 20% (weight in g/volume in L, referred to as w/v). For example, sucrose is preferably present in a concentration of 5 to 15% (w/v).

The modified poxvirus composition, and especially liquid composition thereof, may further comprise a pharmaceutically acceptable chelating agent for improving stability. The pharmaceutically acceptable chelating agent may notably be selected from ethylenediaminetetraacetic acid (EDTA), 1,2-bis(o-aminophenoxy)ethane-N,N,N′,N′-tetraacetic acid (BAPTA), ethylene glycol tetraacetic acid (EGTA), dimercaptosuccinic acid (DMSA), diethylene triamine pentaacetic acid (DTPA), and 2,3-Dimercapto-1-propanesulfonic acid (DMPS). The pharmaceutically acceptable chelating agent is preferably present in a concentration of at least 50 μM with a specific preference for a concentration of 50 to 1000 μM. Preferably, said pharmaceutically acceptable chelating agent is EDTA present in a concentration close to 150 μM.

Additional compounds may further be present to increase stability of the modified poxvirus composition. Such additional compounds include, without limitation, C₂-C₃ alcohol (desirably in a concentration of 0.05 to 5% (volume/volume or v/v)), sodium glutamate (desirably in a concentration lower than 10 mM), non-ionic surfactant (U.S. Pat. No. 7,456,009, US2007-0161085) such as Tween 80 (also known as polysorbate 80) at low concentration below 0.1%. Divalent salts such as MgCl₂ or CaCl₂ have been found to induce stabilization of various biological products in the liquid state (see Evans et al. 2004, J Pharm Sci. 93:2458-75 and U.S. Pat. No. 7,456,009). Amino acids, in particular histidine, arginine and/or methionine, have been found to induce stabilization of various viruses in the liquid state (see WO2016/087457).

The presence of high molecular weight polymers such as dextran or polyvinylpyrrolidone (PVP) is particularly suited for freeze-dried compositions obtained by a process involving vacuum drying and freeze-drying and the presence of these polymers assists in the formation of the cake during freeze-drying (see e.g. WO03/053463; WO2006/085082; WO2007/056847; WO2008/114021 and WO2014/053571).

In accordance with the present invention, the formulation of the composition can also be adapted to the mode of administration to ensure proper distribution or delayed release in vivo. Biodegradable and biocompatible polymers can be used, such as ethylene vinyl acetate, polyanhydrides, polyglycolic acid, collagen, polyorthoesters, polylactic acid and polyethylene glycol. (see e.g. J. R. Robinson in “Sustained and Controlled Release Drug Delivery Systems”, ed., Marcel Dekker, Inc., New York, 1978; WO01/23001; WO2006/93924; WO2009/53937).

For illustrative purposes, Tris-buffered formulations (Tris-HCl pH8) comprising saccharose 5% (w/v), sodium glutamate 10 mM, and NaCl 50 mM are adapted to the preservation of the composition described herein from −20° C. to 5° C.

Dosage

In a preferred embodiment, the composition is formulated in individual doses, each dose containing from about 10³ to 10¹² vp (viral particles), iu (infectious unit) or pfu (plaque-forming units) of the modified poxvirus depending on the quantitative technique used. The quantity of virus present in a sample can be determined by routine titration techniques, e.g. by counting the number of plaques following infection of permissive cells (e.g. HeLa cells) to obtain a plaque forming units (pfu) titer, by measuring the A260 absorbance (vp titers), or still by quantitative immunofluorescence, e.g. using anti-virus antibodies (iu titers). Further refinement of the calculations necessary to adapt the appropriate dosage for a subject or a group of subjects may be routinely made by a practitioner, in the light of the relevant circumstances. As a general guidance, individual doses which are suitable for the poxvirus composition comprise from approximately 10³ to approximately 10¹² pfu, advantageously from approximately 10⁴ pfu to approximately 10¹¹ pfu, preferably from approximately 10⁵ pfu to approximately 10¹⁰ pfu; and more preferably from approximately 10⁶ pfu to approximately 10⁹ pfu and notably individual doses of approximately 10⁶, 5×10⁶, 10⁷, 5×10⁷, 10⁸ or 5×10⁸ pfu are particularly preferred.

Administration

Any of the conventional administration routes is applicable in the context of the invention including parenteral, topical or mucosal routes. Parenteral routes are intended for administration as an injection or infusion and encompass systemic as well as local routes. Parenteral injection types that may be used to administer the poxvirus composition include intravenous (into a vein), intravascular (into a blood vessel), intra-arterial (into an artery such as hepatic artery), intradermal (into the dermis), subcutaneous (under the skin), intramuscular (into muscle), intraperitoneal (into the peritoneum) and intratumoral (into a tumor or its close vicinity) and also scarification. Administration can be in the form of a single bolus dose, or may be, for example, by a continuous perfusion pump. Mucosal administrations include without limitation oral/alimentary, intranasal, intratracheal, intrapulmonary, intravaginal or intra-rectal route. Topical administration can also be performed using transdermal means (e.g. patch and the like). Preferably, the modified poxvirus composition is formulated for intravenous or intratumoral administration in the tumor or at its close vicinity).

Administrations may use conventional syringes and needles (e.g. Quadrafuse injection needles) or any compound or device available in the art capable of facilitating or improving delivery of the modified poxvirus in the subject (e.g. electroporation for facilitating intramuscular administration). An alternative is the use of a needleless injection device (e.g. Biojector™ device). Transdermal patches may also be envisaged.

The composition described herein is suitable for a single administration or a series of administrations. It is also possible to proceed via sequential cycles of administrations that are repeated after a rest period. Intervals between each administration can be from three days to about six months (e.g. 24 h, 48 h, 72 h, weekly, every two weeks, monthly or quarterly, etc). Intervals can also be irregular. The doses can vary for each administration within the range described above. A preferred therapeutic scheme involves 2 to 10 weekly administrations possibly followed by 2 to 15 administrations at longer intervals (e.g. 3 weeks) of the poxvirus composition.

Methods for Using the m2-Defective Poxvirus and Composition of the Invention

In another aspect, the composition described herein is for use for treating or preventing a proliferative disease according to the modalities described herein. Accordingly, the invention also provides a method of treatment comprising administering said composition to a subject in need thereof (preferably a subject afflicted with a cancer) in an amount sufficient to treat or prevent such a disease as well as a method for inhibiting tumor cell growth comprising administering the composition to the subject. In the context of the invention, the methods and use described herein aim at slowing down, curing, ameliorating or controlling the occurrence or the progression of the proliferative disease.

As used herein, the term “proliferative disease” encompasses a broad group of various diseases resulting from uncontrolled cell growth and spread including cancers as well as diseases associated to an increased osteoclast activity (e.g. rheumatoid arthritis, osteoporosis, etc) and cardiovascular diseases (e.g. restenosis that results from the proliferation of the smooth muscle cells of the blood vessel wall). Unregulated cell division and growth may result in the formation of malignant tumors that invade neighbouring tissues and can also metastasize to distant parts of the body through the lymphatic system or bloodstream. The term “cancer” may be used interchangeably with any of the terms “tumor”, “malignancy”, “neoplasm”, etc., and are meant to include any type of tissue, organ or cell, any stage of malignancy (e.g. from a prelesion to stage IV) and encompass solid tumors and blood borne tumors as well as primary and metastatic cancers.

Representative examples of cancers that may be treated using the composition and methods of the invention include, without limitation, carcinoma, lymphoma, blastoma, sarcoma, and leukemia and more particularly bone cancer, gastrointestinal cancer, liver cancer, pancreatic cancer, gastric cancer, colorectal cancer, esophageal cancer, oro-pharyngeal cancer, laryngeal cancer, salivary gland carcinoma, thyroid cancer, lung cancer, cancer of the head or neck, skin cancer, squamous cell cancer, melanoma, uterine cancer, cervical cancer, endometrial carcinoma, vulvar cancer, ovarian cancer, breast cancer, prostate cancer, cancer of the endocrine system, sarcoma of soft tissue, bladder cancer, kidney cancer, glioblastoma and various types of the central nervous system (CNS), etc. In one embodiment the methods and use according to the present invention is for treating a cancer selected from the group consisting of renal cancer (e.g. clear cell carcinoma), prostate cancer (e.g. hormone refractory prostate adenocarcinoma), breast cancer (e.g. metastatic breast cancer), colorectal cancer, lung cancer (e.g. non-small cell lung cancer), liver cancer (e.g. hepatocarcinoma), gastric cancer, bile duct carcinoma, endometrial cancer, pancreatic cancer and ovarian cancer.

Typically, the administration of the composition described herein provides a therapeutic benefit to the treated subject which can be evidenced by an observable improvement of the clinical status over the baseline status or over the expected status if not treated. An improvement of the clinical status can be easily assessed by any relevant clinical measurement typically used by physicians or other skilled healthcare staff. In the context of the invention, the therapeutic benefit can be transient (for one or a couple of months after cessation of administration) or sustained (for several months or years). As the natural course of clinical status which may vary considerably from a subject to another, it is not required that the therapeutic benefit be observed in each subject treated but in a significant number of subjects (e.g. statistically significant differences between two groups can be determined by any statistical test known in the art, such as a Tukey parametric test, the Kruskal-Wallis test the U test according to Mann and Whitney, the Student's t-test, the Wilcoxon test, etc).

For instance, a therapeutic benefit in a subject afflicted with a cancer can be evidenced, e.g., by a reduction in the tumor number, a reduction of the tumor size, a reduction in the number or extent of metastases, an increase in the length of remission, a stabilization (i.e. not worsening) of the state of disease, a decrease of the rate of disease progression or its severity, a prolonged survival, a better response to the standard treatment, an amelioration of the disease's surrogate markers, an improvement of quality of life, a reduced mortality, and/or prevention of the disease's recurrence, etc.

The appropriate measurements such as blood tests, analysis of biological fluids and biopsies as well as medical imaging techniques can be used to assess a clinical benefit. They can be performed before the administration (baseline) and at various time points during treatment and after cessation of the treatment. Such measurements are evaluated routinely in medical laboratories and hospitals and a large number of kits is available commercially (e.g. immunoassays, quantitative PCR assays).

A preferred embodiment is directed to a composition comprising a modified poxvirus, desirably an oncolytic modified poxvirus and preferably an oncolytic vaccinia virus (e.g. Copenhagen strain) with a specific preference for an oncolytic vaccinia virus encoding an anti-CTLA-4 antibody as described herein for use for treating a subject with a cancer, and preferably a renal cancer, a colorectal cancer, a lung cancer (e.g. non-small cell lung cancer), a melanoma and an ovarian cancer.

In another embodiment, the modified poxvirus or composition described herein is for use for enhancing an anti-tumoral adaptative immune response or for enhancing or prolonging an antitumor response.

In another aspect, the modified poxvirus or composition thereof is used or administered for stimulating or improving an immune response in the treated subject. Accordingly, the present invention also encompasses a method for stimulating or improving an immune response comprising administering the composition to the subject in need thereof, in an amount sufficient according to the modalities described herein so as to stimulate or improve the subject's immunity. The stimulated or improved immune response can be specific (i.e. directed to epitopes/antigens) and/or non-specific (innate), humoral and/or cellular, notably a CD4+ or CD8+-mediated T cell response. The ability of the composition described herein to stimulate or improve an immune response can be evaluated either in vitro (e.g. using biological samples collected from the subject) or in vivo using a variety of direct or indirect assays which are standard in the art (see for example Coligan et al., 1992 and 1994, Current Protocols in Immunology; ed J Wiley & Sons Inc, National Institute of Health or subsequent editions). Those cited above in connection with the antigenic nature of a polypeptide are also appropriate.

In particular and compared to a conventional (m2-positive) poxvirus, the modified poxvirus or composition described herein is also useful for any of the following purposes or any combination thereof:

for stimulating or improving a lymphocyte-mediated immune response (especially against an antigenic polypeptide);

for stimulating or improving the activity of APC;

for stimulating or improving an anti-tumoral response;

for stimulating or improving the CD28-signalling pathway;

for improving the therapeutic efficacy provided by the modified poxvirus described herein in a treated subject or a group of treated subjects; and/or

for reducing the toxicity provided by the modified poxvirus described herein in a treated subject or a group of treated subjects.

Combination Therapy

In one embodiment, the modified poxvirus, composition or methods of the invention are used as stand-alone therapy. In another embodiment, they can be used or carried out in conjunction with one or more additional therapies, in particular standard of care therapy(ies) that are appropriate for the type of cancer afflicting the treated subject. Standard-of-care therapies for different types of cancer are well known by the person skilled in the art and usually disclosed in Cancer Network and clinical practice guidelines. Such one or more additional therapy(ies) are selected from the group consisting of surgery, radiotherapy, chemotherapy, cryotherapy, hormonal therapy, toxin therapy, immunotherapy, cytokine therapy, targeted cancer therapy, gene therapy, photodynamic therapy and transplantation, etc.

Such additional anticancer therapy/ies is/are administered to the subject in accordance with standard practice before, after, concurrently or in an interspersed manner with the modified poxvirus or composition described herein. Concurrent administration of two or more therapies does not require that the agents be administered at the same time or by the same route, as long as there is an overlap in the time period during which the composition and additional anti-cancer therapy are exerting their therapeutic effect. Concurrent administration includes administering the modified poxvirus composition within the same day (e.g. 0.5, 1, 2, 4, 6, 8, 10, 12 hours) as the other therapeutic agent. Although any order is contemplated by the present invention, it is preferred that the modified poxvirus composition be administered to the subject before the other therapeutic agent.

In specific embodiments, the modified poxvirus or composition described herein may be used in conjunction with surgery. For example, the composition may be administered after partial or total surgical resection of a tumor (e.g. by local application within the excised zone, for example).

In other embodiments, the modified poxvirus or composition described herein can be used in association with radiotherapy. Those skilled in the art can readily formulate appropriate radiation therapy protocols and parameters (see for example Perez and Brady, 1992, Principles and Practice of Radiation Oncology, 2nd Ed. JB Lippincott Co; using appropriate adaptations and modifications as will be readily apparent to those skilled in the field). The types of radiation that may be used notably in cancer treatment are well known in the art and include electron beams, high-energy photons from a linear accelerator or from radioactive sources such as cobalt or cesium, protons, and neutrons. Dosage ranges for radioisotopes vary widely, and depend on the half-life of the isotope, the strength and type of radiation emitted, and the uptake by the neoplastic cells. Regular X-rays doses for prolonged periods of time (3 to 6 weeks), or high single doses are contemplated by the present invention.

In certain embodiments, the modified poxvirus or composition described herein may be used in conjunction with chemotherapy. Representative examples of suitable chemotherapy agents currently available for treating cancer include, without limitation, alkylating agents, topoisomerase I inhibitors, topoisomerase II inhibitors, platinum derivatives, inhibitors of tyrosine kinase receptors, cyclophosphamides, antimetabolites, DNA damaging agents and antimitotic agents. Representative examples of suitable chemotherapy agents currently available for treating infectious diseases include among other antibiotics, antimetabolites, antimitotics and antiviral drugs (e.g. interferon alpha).

In further embodiments, the modified poxvirus or composition described herein may be used in conjunction with immunotherapeutics such as anti-neoplastic antibodies as well as siRNA and antisense polynucleotides.

In still further embodiments, the modified poxvirus or composition described herein may be used in conjunction with adjuvant. Representative examples of suitable adjuvants include, without limitation, TLR3 ligands (Claudepierre et al., 2014, J. Virol. 88(10): 5242-55), TLR9 ligands (e.g. Fend et al., 2014, Cancer Immunol. Res. 2, 1163-74; Carpentier et al., 2003, Frontiers in Bioscience 8, e115-127; Carpentier et al., 2006, Neuro-Oncology 8(1): 60-6; EP 1 162 982; U.S. Pat. Nos. 7,700,569 and 7,108,844) and PDE5 inhibitors such as sildenafil (U.S. Pat. Nos. 5,250,534, 6,469,012 and EP 463 756).

In additional embodiments, the modified poxvirus or composition described herein may be used according to a prime boost approach which comprises sequential administrations of a priming composition(s) and a boosting composition(s). Typically, the priming and the boosting compositions use different vectors which encode at least an antigenic domain in common with at least one being the modified poxvirus described herein. Moreover, the priming and boosting compositions can be administered at the same site or at alternative sites by the same route or by different routes of administration.

Other features, objects, and advantages of the invention will be apparent from the description and drawings and from the claims. The following examples are incorporated to illustrate preferred embodiments of the invention. However, in light of the present disclosure, those skilled in the art should appreciate that changes can be made in the specific embodiments that are disclosed without departing from the spirit and scope of the invention.

### EXAMPLES

Material and Methods

Proteins and Viruses

Recombinant Fc fusion proteins (human and murine) with or without a His-tag at their C-terminus were ordered at R&D Systems. Human CD80-Fc and CD86-Fc were biotinylated in house using Biotinamidohexanoyl-6-aminohexanoic acid N-hydroxysuccinimide ester (Sigma).

Various vaccinia viruses were used:
Wild type vaccinia (Copenhagen, Wyeth and Western Reserve strains);
Double deleted vaccinia viruses (Copenhagen strain) defective for both thymidine kinase and ribonucleotide reductase activities (tk-; rr-; described in WO2009/065546).
Triple deleted vaccinia viruses (Copenhagen strain) defective for tk, rr- and m2 activities. The triple deleted virus was generated from the double deleted tk-rr-by specific homologous recombination into the open reading frame of the M2L locus as described hereinafter.

Beside vaccinia virus, all poxviruses tested, unless otherwise specified, were wild type strains.

Deletion of M2L in Rr-, Tk-Vaccinia Virus

For in vivo studies, double (tk-rr-) and triple (tk-rr-m2-) deleted vaccinia viruses were engineered to encode the firefly luciferase at the J2R locus under the p11K7.5 promoter.

The M2L gene deletion introduced into the VV genome encompasses 64 nucleotides upstream m2 ORF and the 169 first codons of the m2 ORF. The deletion was performed by homologous recombination using a transfer plasmid from PUC18 origin. This transfer plasmid contained a left (nt26980 to 27479 of VV genome Accession M35027) and a right (nt28051 to 28550) arm separated by an expression cassette encoding the fusion of selection markers enhanced green fluorescent protein/xanthine-guanine phosphoribosyl transferase (EGFP/GPT) under vaccinia pH5R promoter control. The resulting plasmid was transfected by electroporation into Chicken embryo fibroblasts (CEF) infected by vaccinia virus encoding luciferase (rr-; tk-/luciferase) using the Amaxa Nucleofactor. The recombinant virus was isolated by EGFP/GPT selection. The deletion of M2L and insertion of the EGFP/GPT cassette were confirmed by PCR analysis. The EGFP/GPT selection cassette was removed by passing the recombinant virus on CEFs without selection. A primary research stock was produced on CEFs. The deletion of the M2L gene was verified by PCR and sequencing.

The virus was produced on CEF after infection at MOI 0.05 and three days of incubation. Three days after infections, the crude harvest containing infected cells and culture supernatant was recovered and stored at −20° C. until use. Prior to purification, this suspension was homogenized in order to release viral particles. Large cellular debris were then eliminated by depth filtration. The clarified viral suspension was subsequently concentrated and diafiltered with the formulation buffer by using tangential flow filtration and size hollow fiber microfiltration filters. Finally, the purified virus was further concentrated using the same tangential flow filtration system, aliquoted and stored at −80° C. until use.

ELISA Assay for B7 Binding

Ninety-six well plates (Nunc immune plate Medisorp) were coated, overnight at 4° C., with 100 μL of 0.5 μg/mL of either B7, CTLA4 or CD28 proteins in coating buffer (50 mM Na carbonate pH 9.6). Microplates were washed by PBS/0.05% Tween 20 and saturated by 200 μL of blocking solution (PBS; 0.05% Tween 20; 5% Non-Fat Dry Milk (Biorad)). All antibody preparations and dilutions were made in blocking solution. One hundred μL of samples were added to each well in triplicate and in two-fold serial dilutions for some experiments (binding curves). Microplates were then incubated with 100 μL of anti-Flag-HRP (Sigma) diluted 10 000-fold. Microplates were then incubated with 100 μL/well of 3,3′,5,5′-tétraméthylbenzidine (TMB, Sigma) and reaction was stopped with 100 μL 2M H₂SO₄. Absorbance was measured at 450 nm with a plate reader (TECAN Infinite M200PRO). The absorbance values were transferred into the software GraphPadPrism for analysis and graphic representation.

Competition ELISA

Experimental conditions and solution, not otherwise specified, were identical to the ones described above. For CTLA4/CD80, CD28/CD80 and PDL1/CD80 competition assays, 100 μL of CTLA4, CD28 and PDL1 were coated at 0.25 (CTLA4) or 1 μg/mL (CD28 and PD-L1). Samples were added and diluted (two-fold serial dilution) in blocking solution containing constant concentration of CD80 (either 50, 250 or 500 ng/mL for CTLA4, CD28 and PD-L1 respectively). For CD86/CTLA4 and CD28/CD86 competition essays, 100 μL of CD86 or CD28 were coated at 0.25 (CD86) or 2 μg/mL (CD28). Samples were added and diluted (two-fold serial dilution) in blocking solution containing constant concentration of either CTLA4 (100 ng/mL) or CD86 (500 ng/mL) respectively. Either anti-His tag-HRP (Qiagen) at 1/2000 or streptavidin HRP (Southern Biotech) at 1/1000 were used as conjugated reagents. The plates were further treated, and results analysed, as described above.

Western Blot

Twenty-five microliters of samples were prepared in Laemmli buffer containing 5% β-mercaptoethanol (BME) (reducing condition) or not (non-reducing condition). After electrophoresis on Criterion TGX 4-15% stain free gel (Biorad) the proteins were transferred to PVDF membrane (Transblot Turbo System). IBind Flex Western system (Invitrogen) was used for the proteins/antibodies incubations and washes. Blots were probed with 2.5 μg/mL CD80-Fc, CD86-Fc, CTLA4-Fc or anti-Flag-HRP at 1/1000. For CD80-Fc, CD86-Fc and CTLA4-Fc a HRP anti-Human Fc (Bethyl) at 1/3000 was used as conjugated antibody. The 1× iBind Flex Solution was used to block, dilute the antibodies, wash and wet the iBind Flex Card. Immune complexes were detected using the Amersham ECL Prime Western Blotting reagents. Chemiluminescence was recorded with a Molecular Imager ChemiDOC XRS (Biorad).

Affinity Chromatography

Supernatants of CEF infected (MOI 0.05) by either MVA or vaccinia virus Copenhagen were collected 72 hours post-infection. The supernatants were centrifugated and filtered on 0.2 μm filters to remove most of cellular debris and vaccinia virus. The treated supernatants supplemented with 0.05% Tween 20 were then concentrated ˜20-fold using vivaspin 20 30 000 MWCO cut-off concentrator (Sartorius). Streptavidin magnetic beads (GE healthcare) were coated with either an irrelevant monoclonal biotinylated antibody (chCXIIG6), CTLA4-Fc-Biot or CD86-Fc-Biot. Four mL of concentrated supernatants (MVA and Vaccinia virus Copenhagen) were incubated with 24 μL of chCXIIG6-Streptavindin beads to remove unspecific binding. The flow-throughs of this first incubation were split in 2 equal parts and incubated either with CTLA4-Fc-Biot-streptavidin beads or CD86-Fc-Biot-streptavidin beads to yield the four following arms: MVA supernatant+CTLA4 beads (MVA A4); MVA supernatant+CD86 beads (MVA CD); Vaccinia virus+CTLA4 beads supernatant (VV A4) and Vaccinia virus+CD86 beads (VV CD86). The beads were extensively washed with PBS, 0.05% Tween 20 followed by PBS and bound proteins were eluted two times by 50 μL 0.1M acetic acid neutralized immediately by addition of 4 μL 2M Tris Base. The two elutions were then pooled before MS analysis.

Protein Preparation for Digestion.

Ten or 20 μl of sample were evaporated and submitted to reduction by solubilization in 10 μl of 10 mM DTT in 25 mM NH4HCO3 (1H at 57° C.). Reduced cysteine residues were alkylated 10 μl of 55 mM iodoacetamide in 25 mM NH4HCO3 30 min at room temperature in the dark. The trypsin (12.5 ng/μL; Promega V5111) freshly diluted in 25 mM NH4HCO3 was added to the sample in a 1:100 (enzyme/protein) ratio to a final volume of 30 μl and incubated 5 hours at 37° C. The activity of the trypsin is inhibited by acidification with 5 μl of H2O/TFA 5%.

MS/MS analysis.

Samples were analysed on a nanoUPLC-system (nanoAcquity, Waters) coupled to a quadrupole-Orbitrap hybrid mass spectrometer (Q-Exactive plus, Thermo Scientific, San Jose, Calif.). The UPLC system was equipped with a Symmetry C18 precolumn (20×0.18 mm, 5 μm particle size, Waters, Milford, USA) and an ACQUITY UPLC® BEH130 C18 separation column (75 μm×200 mm, 1.7 μm particle size, Waters). The solvent system consisted of 0.1% formic acid in water (solvent A) and 0.1% formic acid in acetonitrile (solvent B). Two μL of each sample were injected. Peptides were trapped during 3 min at 5 μL/min with 99% A and 1% B. Elution was performed at 60° C. at a flow rate of 400 μL/min, using a 79 minutes linear gradient from 1-35% B. To minimize carry-over, a column wash (50% ACN during 20 min.) was included in between each sample in addition to a solvent blank injection, which was performed after each sample.

The Q-Exactive Plus was operated in positive ion mode with source temperature set to 250° C. and spray voltage to 1.8 kV. Full scan MS spectra (300-1800 m/z) were acquired at a resolution of 140,000 at m/z 200, a maximum injection time of 50 ms and an AGC target value of 3×106 charges with the lock-mass option being enabled (445.12002 m/z). Up to 10 most intense precursors per full scan were isolated using a 2 m/z window and fragmented using higher energy collisional dissociation (HCD, normalised collision energy of 27 eV) and dynamic exclusion of already fragmented precursors was set to 60 sec. MS/MS spectra were acquired with a resolution of 17,500 at m/z 200, a maximum injection time of 100 ms and an AGC target value of 1×105. The system was fully controlled by the XCalibur software (v3.0.63; Thermo Fisher Scientific).

MS/MS Data Interpretation

MS/MS data were searched against a Gallus gallus and Vaccinia virus Uniprot database derived combined target-decoy database (Jan. 4, 2018, containing 33939 target sequences plus the same number of reversed decoy sequences) using Mascot (version 2.5.1, Matrix science, London, England). The targets proteins hCTLA4, hCD86 and hCXIIG6 and target-decoy were manually added to the database. The database including common contaminants (human keratins and porcine trypsin) and was created using an in-house database generation toolbox (http://msda.u-strasbg.fr). The following parameters were applied: one missed cleavage by trypsin and variable modifications (oxidation of Methionine (+16 Da), carbamidomethylation of Cysteine (+57 Da), were considered. The search window was set to 25 ppm for precursor ions and 0.07 Da for fragment ions. Mascot result files (.dat) were imported into Proline software (http://proline.profiproteomics.fr/) and proteins were validated on pretty rank equal to 1, 1% FDR on peptide spectrum matches based on adjusted e-value, at least 1 specific peptide per protein, 1% FDR on protein sets and Mascot Modified Mudpit scoring.

Mixed Lymphocyte Reaction (MLR)

The capacity of the m2-virus to activate lymphocytes was evaluated in MLR assays. CEF cells were infected (MOI 0.05) by either COPTG19289, VVTG18058 or MVAN33 and culture supernatants were harvested 48 h post-infection and concentrated ˜20-fold using vivaspin 20 30000 MWCO cut-off concentrator (Sartorius). The concentrated supernatants were added (20 μL in 200 μL) either undiluted or diluted 10 and 100-fold to yield a final “supernatant concentration” of 2, 0.2 and 0.02-fold respectively.

Blood from different healthy donors were purchased at Etablissement Français du sang (EFS Grand Est, 67065 Strasbourg). PBMC were purified by Ficoll-Paque method (Ficoll-Paque PLUS, GE Healthcare) and resuspended at about 1×10⁷ cells/mL in RPMI medium supplemented with 20% FBS (Fetal bovine serum) and 10% DMSO and stored at −150° C. until use. PBMC were thawed at 37° C., resuspended in RMPI medium with 10% FBS and centrifugated 5 minutes at 300 g. Collected cells were resuspended in RMPI medium+10% FBS and cell concentration was adjusted to 3×10⁶ cells/mL. One hundred μL of PBMC from two different donors were mixed in a well of a 96-well microplate in triplicate. Twenty μL of infected cell supernatants described above were added to each well and the microplates were incubated 72 h at 37° C. in an atmosphere of 5% CO₂.

The culture supernatants of the MLR were then harvested and the human Interleukin-2 (IL-2) measured by ELISA using the human IL-2*-2ELISA MAX™ deluxe Set kit (BioLegend). The measures were normalized by dividing the mean of IL-2 concentration of the three replicates of a given sample by the mean of IL-2 concentration of the three replicates of PBMC incubated with medium.

In Vivo Experiments in Humanized NCG-34+ Mice

Mice Humanization

The NOD/Shi-scid/IL-2Rγnull immunodeficient mouse strain (NCG) was provided by Taconic. Four-week-old animals were treated with busulfan intraperitoneally (chemoablation) and injected intravenously (IV) the next day with CD34+ human stem cells (50,000 cells per mouse). Fourteen weeks after cell injection, engraftment level was monitored via the analysis of human CD45+ cells among total blood leukocytes by flow cytometry. Humanization rate was defined as the ratio of circulating hCD45/total CD45 (mCD45+hCD45).

T Lymphocytes Immune Phenotype

Blood (100 μL) was collected from the retro-orbital sinus 2 days before tumor engraftment. Human CD45+, CD3+, CD3+CD4+ and CD3+CD8+ lymphocyte populations were assessed by flow cytometry (Attune NxT, Lifetechnologies) using antibodies directed against hCD45 (Ref 563879; BD), CD4 (Ref 130-092-373; Miltenyi), CD3 (Ref 130-109-462; Miltenyi) and CD8 (Ref 130-096-561; Miltenyi) as well as a live/dead yellow marker (Ref L34968; Thermofisher). Briefly, blood samples were incubated with the various antibodies during 30 min at 4° C. Then, red blood cells were lysed using High Yield Lysis buffer (HYL250; Thermo Fischer Scientific) at room temperature (RT) for 15 min, directly followed by flow cytometry analysis (Attune NxT, Life technologies).

Treatment with Oncolytic Viruses

Human colorectal carcinoma cells HCT-116 were purchased from ATCC (CCL-247™), grown in McCoy's 5A medium supplemented with 10% FBS+Penicillin/Streptomycin and detached with Trypsin at 37° C. for 10 min. After washing, cells were re-suspended in sterile PBS at 5×10⁷ cells/ml and 100 μl of the cell suspension (5×10⁶ cells) were injected subcutaneously in one flank of the mice. When the average tumor volume almost reached 70 mm³, mice were randomized into five groups (5 mice/group) based on their humanization rate and tumor size:
Group 1 received vehicle
Group 2 received 10⁵ pfu of VVTG18058
Group 3 received 10⁶ pfu of VVTG18058
Group 4 received 10⁵ pfu of COPTG19289
Group 5 received 10⁶ pfu of COPTG19289

For each group, a single intravenous (IV) injection of 100 ul of the viral preparation was performed the day of randomization, defined as D0. Mice were monitored daily for unexpected signs of distress. Body weight and tumor volume were monitored 3 times per week. Tumor diameters were measured using a caliper. Tumor volumes (in mm³) were calculated according to the following formula: Volume=½ (length×width²). Animal were sacrificed when tumor volume exceeds 1500 mm³ or when body weight loss is above 25%.

### Example 1: Identification of the Ability of the Vaccinia Virus m2 Protein to Interfere with B7-Mediated Costimulatory Pathway and Characterization of its Binding Properties

Supernatants of Vaccinia Virus Infected Cell Inhibits the Interaction of CTLA4 with CD80 or CD86

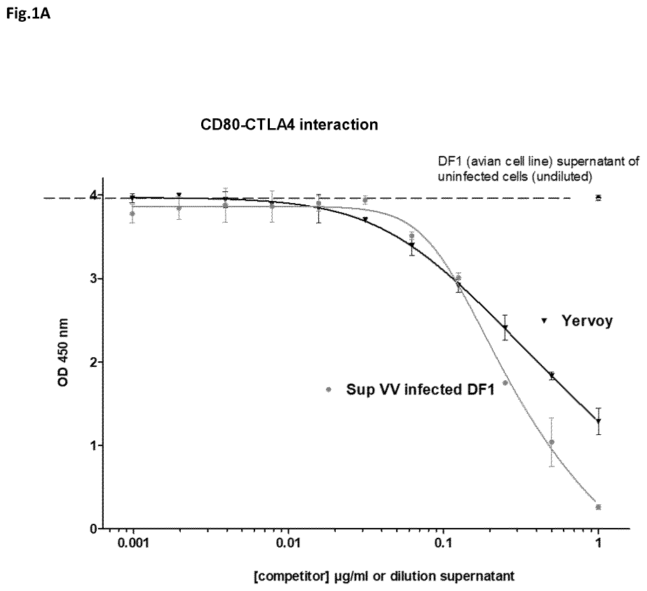

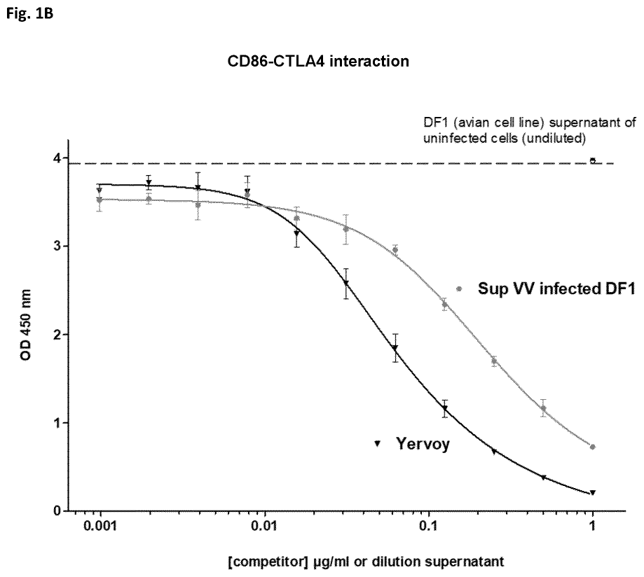

Two assays were set-up to monitor quantitatively the CD80/CTLA4 and CD86/CTLA4 blocking activities provided by the different virus candidates. In these assays, human CTLA4 (hCTLA4) was immobilized on ELISA plate and soluble tagged hCD80 or hCD86 were added. In this setting, any competitive molecule that binds to either the immobilized or the soluble partner will induce a decrease of signal (competition assay). The anti-hCTLA4 antibody Ipilimumab (Yervoy) and supernatant of uninfected DF1 (chicken cells line available; e.g. from ATCC® CRL-12208™) were used as positive and negative controls, respectively. Surprisingly, as Yervoy which interacts with the coated hCTLA-4, all supernatants of cells infected by vaccinia virus (Copenhagen, Wyeth and Western Reserve strains) were found to be competitive in dose-response manner with both CD80/CTLA4 and CD86/CTLA4 assays (FIGS. 1A and 1B), whereas the supernatant of the uninfected DF1 cells did not have any effect. Interestingly, the supernatants of DF1 infected by modified vaccinia virus Ankara (MVA) were not producing any inhibition of the hCTLA4/hCD80 and hCTLA4/hCD86 interactions indicating that this interference ability is not conserved in this virus which has lost six genomic fragments (deletions I to VI) during its attenuation process (data not shown). These results suggest that something in VV supernatants interfered with the binding of CTLA-4 with CD80 and CD86.

To rule out any artefact involving cell or medium components, different cells lines from different origins (avian primary and human tumoral cell lines) were tested and a method of FACS competition was also assayed.

Competition FACS analysis was carried out using a human cell line (i.e. KM-H2, Hodgkin lymphoma) displaying naturally hCD80 and hCD86 at its surface. Binding of soluble recombinant CTLA4-Fc to KM-H2 cells was shown using a fluorochrome-conjugated anti-Fc antibody. When co-incubated with CTLA4-Fc, supernatants of vaccinia virus-infected cells competed for CTLA4-Fc binding to KM-H2 cells in marked contrast to MVA-infected cells which behave as the negative control (data not shown).

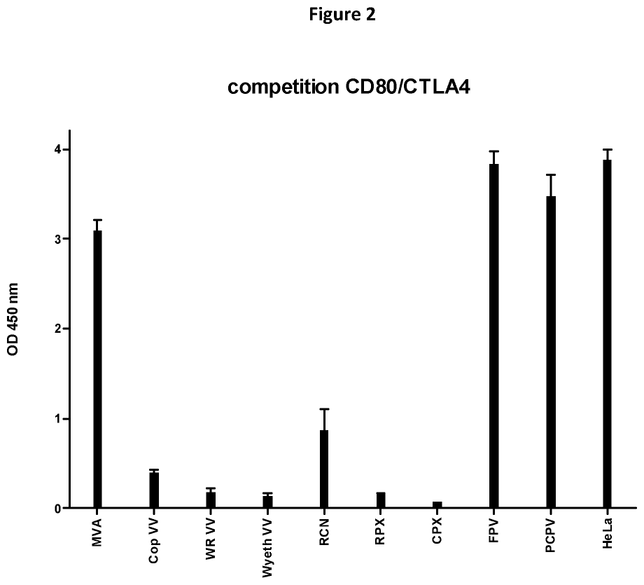

Competition ELISA was carried out using supernatants of HeLa (instead of DF1) cells infected with different poxviruses to evaluate their capacity to interfere with the CTLA4 binding to CD80 or CD86. Various strains of vaccinia virus (Wyeth, WR and Copenhagen) were tested as well as other orthopox (e.g., raccoonpox, rabbitpox, cowpox, MVA), avipox (fowlpox) and parapox virus (pseudocowpox virus). Uninfected HeLa cells are used as negative control. In this screening experiment, HeLa cells were infected with different poxviruses at a high MOI (MOI 1) to guarantee an optimal infection and the resulting supernatants were collected and tested by evaluating their capacity to inhibit the CTLA4-Fc binding to CD80 (represented as OD450 nm). As illustrated in FIG. 2, all supernatants of cells infected with either the three strains of vaccinia virus or with raccoonpox (RCN), rabbitpox (RPX) and cowpox (CPX) were able to interfere with the binding of hCTLA4 to hCD80. These results indicate that a factor secreted during infection with these poxviruses was interfering with the CTLA4-B7 pathway. The new unknown factor involved in this inhibitory activity was called “interference factor” (IF). Again, supernatants of cells infected with MVA and some other poxviruses like the pseudocowpox virus (PCPV) and fowlpoxvirus (FPV) did not display any inhibition of the CTLA4/CD80-CD86 interactions as the uninfected HeLa cells (HeLa).

The “Interference Factor” is Present in Vaccinia Virus Supernatants but not in MVA Supernatants

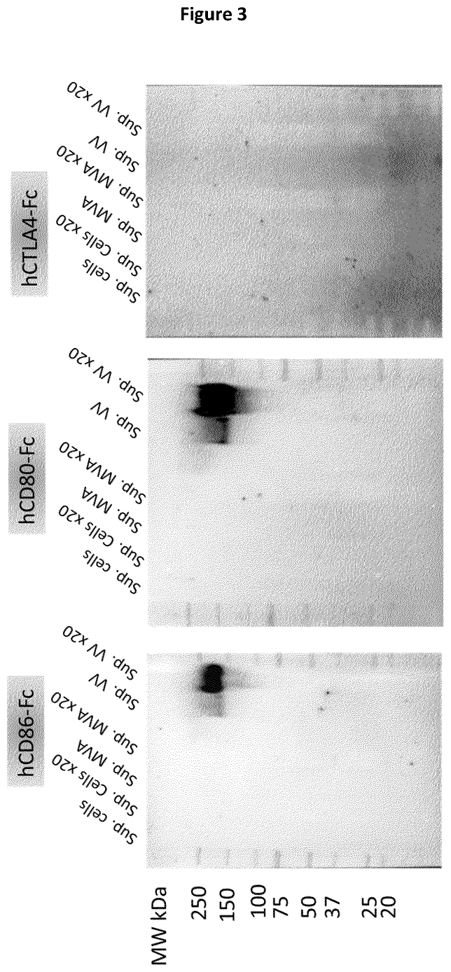

To figure out with which molecule present in VV-infected supernatants, the IF was interacting, a western blot of supernatants of CEF (also designated CEP) uninfected or infected with either MVA or vaccinia virus was probed with the three components of the ELISA assay described above (namely hCD80, hCD86 and hCTLA4). CEF were chosen since they are permissive to both vaccinia virus and MVA that produce, or not, the IF, respectively. Each protein used to probe the western blot was a fusion with an Fc part that allows, among other things, their dimerization and their detection with the same anti-Fc conjugated antibody. Each supernatant was used either as such or concentrated 20 times (×20) The blots presented on FIG. 3 demonstrated unambiguously that a large molecule of about 200 kDa was present only in the vaccinia virus infected supernatants and highlighted with both hCD80 and hCD86 but not with hCTLA4 (at least in these immunoblot conditions). This band is easily detected even in non-concentrated supernatants. Reactivity with both hCD80-Fc and hCD86-Fc was lost in reducing conditions (no detection of any band) indicating that intra and/or inter disulfide bonds are necessary to maintain the IF's structure and interaction with CD80 and CD86 (data not shown). In marked contrast, no band was highlighted in MVA supernatants.

Characterization of the Binding Properties: The “Interference Factor” Present in Vaccinia Virus Supernatant Inhibits the Binding of CD80 and CD86 to CTLA4 and CD28 but Potentializes the Binding of CD80 with PD-L1.

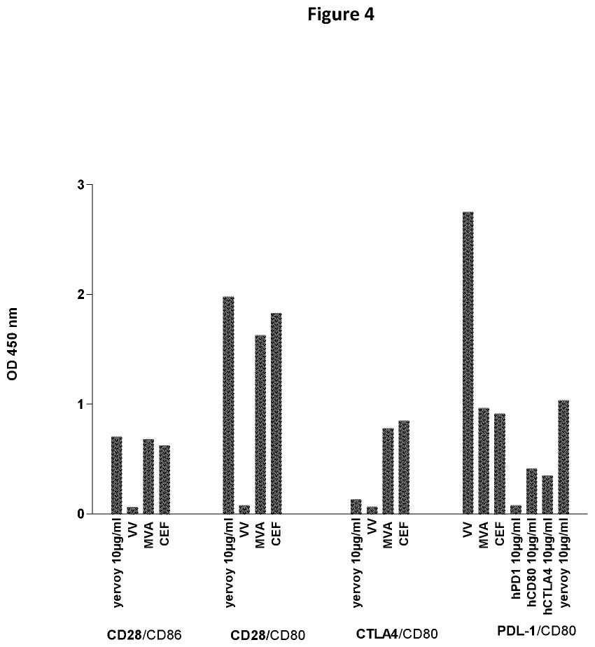

As discussed above, CD80 and CD86 are important co-stimulation antigens involved in the regulation of the adaptive T cell response. Because CD80 and CD86 are involved in several molecular interactions with negative (CTLA4 for both, and PD-L1 for CD80 only) and positive (CD28) outcomes in term of immune response, different ELISAs were set up to decipher the effect of IF on each of these 5 specific interactions. The undiluted supernatants from CEF infected with non-recombinant vaccinia virus (VV) were tested in these different assays and compared to supernatant of MVA-infected CEF and the anti-hCTLA4 antibody Yervoy (10 μg/ml). Supernatants of uninfected CEF cells are used as negative control. As illustrated in FIG. 4, VV supernatant inhibited the interaction of CD80 and CD86 with CTLA4 (as evidenced by an impressive decrease of the OD450 nm absorbance) similarly as Yervoy (as expected due to the binding of Yervoy to its CTLA-4 target that prevents access to CD80 and thus CTLA4/CD80 ligation). In marked contrast, MVA-infected cell supernatants had no effect (same absorbance as the negative CEF control). Moreover, VV supernatants were also able to abolish the positive interaction of CD80 or CD86 with CD28 (strong diminishment of the OD450 nm absorbance with respect to absorbance measured with the supernatant of uninfected CEF cells). In contrast, MVA-infected cell supernatants and Yervoy (as expected for an antibody that target only CTLA4 receptor) had no effect (same absorbance as the negative control). These results confirm the presence of an “IF” in supernatants of VV infected cells whereas MVA genome does not produce such a factor.

Surprisingly, the PD-L1/CD80 interaction was increased by the presence of vaccinia virus supernatants (strong increase of the OD450 nm absorbance with respect to the negative control) reinforcing the PDL1-mediated immunosuppressive signalling. In contrast, Yervoy and MVA-infected CEF supernatants had no impact on PDL1/CD80 (same absorbance as uninfected control). As expected, recombinant hCD80, hCTLA4 and hPD1 abolished this interaction. This result indicates that the IF and CTLA4 binding sites on CD80 are not completely overlapping. It should be noted that the CD80/PD-L1 interaction has been recently involved to Treg survival.

These results highlight the improved immunosuppressive properties displayed by the poxviral m2 polypeptide. Indeed, m2 pushes toward immunosuppressive pathways by blocking CD80/CD28, CD86/CD28 and by potentializing PDL1-CD80 pathways whereas CTLA4-Fc inhibits these three pathways including the immunosuppressive PDL1-CD80 interaction.

Identification of the m2 Poxviral Protein as being the Interference Factor

Based on the apparent molecular weight of approximately 200 kDa and the fact that IF was not present in MVA-infected supernatants, the 37 genes that are different between vaccinia Copenhagen strain and MVA were investigated for a potential candidate without finding any obvious one. No protein of about 200 kDa could be identified. The largest encoded protein, among these 37 gene candidates is the DNA-dependent RNA polymerase subunit rpo147 (J6R) with a theoretical mass of 147 kDa, thus lower than the 200 kDa observed. Based on primary structure, there was no obvious viral protein candidate that could be linked to IF.

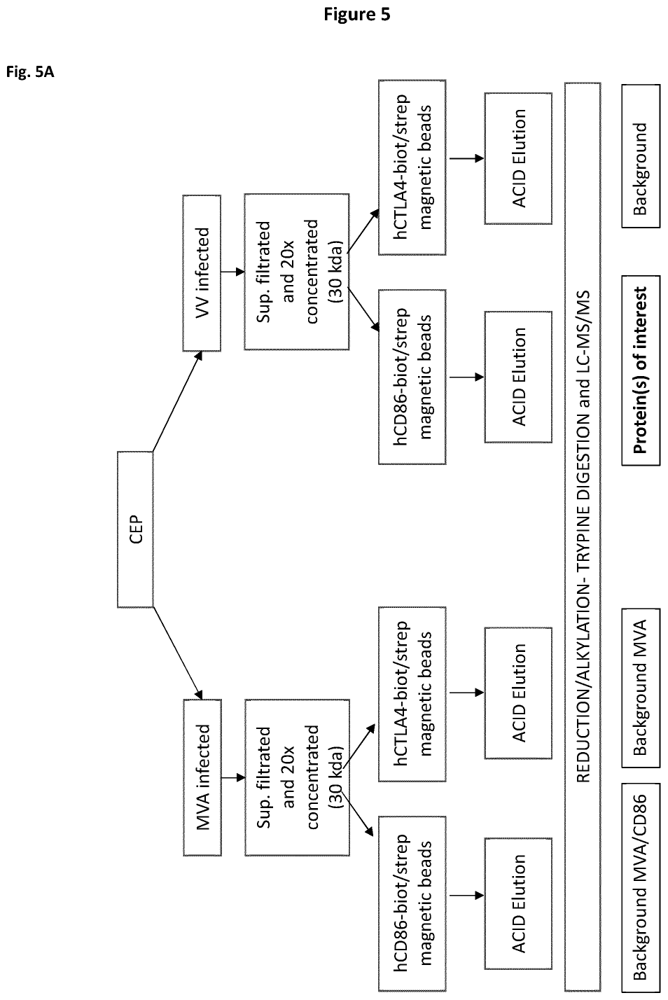

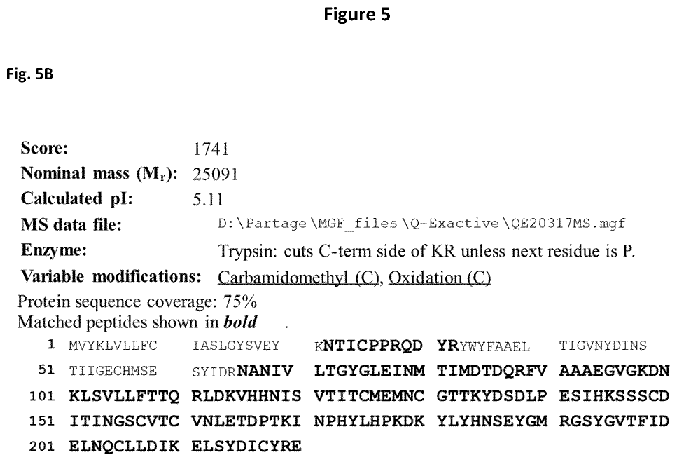

Therefore, an experimental approach to identify IF was attempted using an affinity chromatography (see scheme FIG. 5A) to capture IF. A 20-fold concentrated supernatant of vaccinia virus infected CEF (VV infected) was loaded on this affinity chromatography. A 20-fold concentrated supernatant of MVA infected cells (MVA infected) was processed in parallel. The VV and MVA supernatants were submitted to either immobilized CTLA4 (negative controls) or immobilized CD86-Fc fusion before being eluted by acid. The different elutions of the affinity chromatography arms were analyzed by MS/MS (mass spectrometry) after trypsic digestion. The obtained m/z data were used to probe the chicken (Gallus gallus) and vaccinia virus data banks. One hit was obtained only from the supernatant of vaccinia infected CEF incubated with CD86 coated beads which covers 75% (including the peptide signal) or 82% (without the peptide signal) of the vaccinia virus protein m2 protein encoded by the M2L locus (FIG. 5B where the sequence covered by the detected peptides is indicated in bold). This result is in full agreement with the absence of M2L locus in MVA genome and with the fact that m2 has a predicted signal peptide making it a putative secreted protein.

However, m2 protein has a calculated molecular weight of only 25 kDa and has been reported to migrate on SDS-PAGE on reducing conditions as a 35 kDa protein (Hinthong et al. 2008) which is far from the 200 kDa mass of IF observed on SDS-PAGE. Nevertheless, to our knowledge, the behavior of m2 protein on SDS-PAGE in non-reducing conditions was not documented. Therefore, we hypothesize that IF could be a homo or hetero-multimeric complex involving the VV m2 protein with inter-subunit disulfide bonds resulting in an apparent mass on SDS-PAGE of approximately 200 kDa.

### Example 2: m2 —Defective Poxvirus does not Produce IF Anymore

Construction of M2L-Deleted Poxviruses

The involvement of m2 in the IF was further investigated by deleting the M2L gene in a vaccinia virus genome. Specifically, the M2L locus was disrupted in a double deleted (DD) vaccinia virus expressing the luciferase (i.e. tk⁻, rr-⁻ described in WO2009/065546 and designated VVTG18277) resulting in a recombinant triple deleted (TD) virus (i.e. tk⁻ rr⁻, m2⁻) expressing the luciferase (COPTG19289) as described above. The M2L partial deletion which extends from 64 nucleotides upstream the m2 ORF to the 169 first codons resulted in a suppressed expression of m2 protein (m2-) and did not have any significant impact on the virus replication on CEF compared to the parental one (data not shown).

M2L Deleted Virus does not Produce IF Anymore

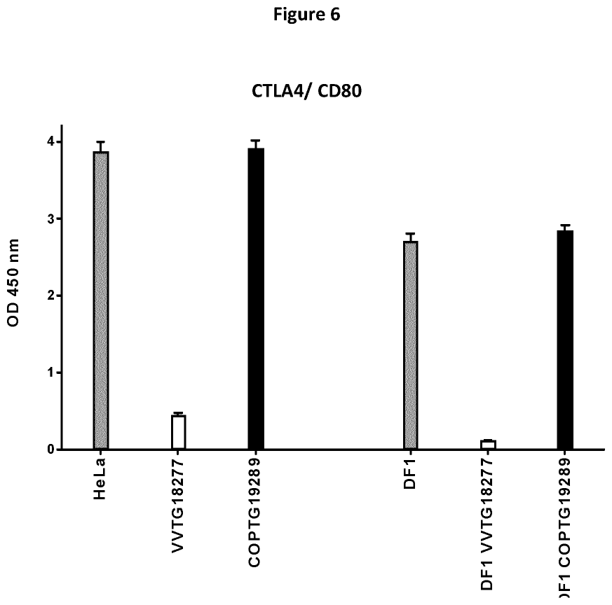

The supernatants obtained upon infection of human HeLa and avian DF1 cells with the DD and TD viruses were studied by competition ELISA as before. As shown in FIG. 6, the supernatant collected upon infection with the M2L-deleted vaccinia virus COPTG19289 was not able anymore to inhibit the CTLA4/CD80 interactions (as evidenced by the same absorbance as the one measured in the uninfected HeLa or DF1 cells) unlike the parental DD virus (VVTG18277) which showed a strong decrease in the absorbance measurement compared to the negative controls.

Moreover, when submitted to western blotting as above, the large complex migrating at 200 kDa detected using CD80-Fc or CD86-Fc probes was no more detected with the supernatant of M2L-deleted virus (data not shown). These results confirmed that m2 is, at least, part of the IF.

### Example 3: m2-Defective Recombinant Poxvirus

Construction of the tk-rr-m2-oncolytic vaccinia virus expressing luciferase (gene inserted into the J2R locus) is described above.

Oncolytic Activity

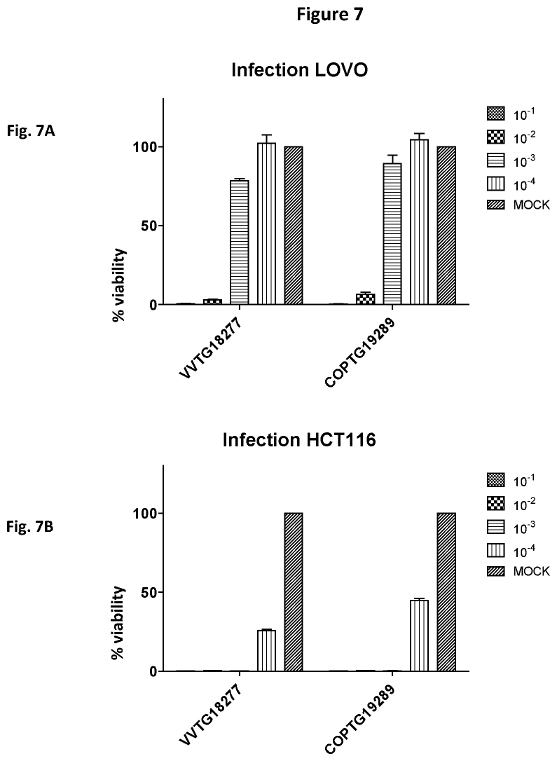

LOVO (ATCC® CCL-229™) and HT116 (ATCC® CCL-247™) colon carcinoma cells were seeded in 96 well plates at a cell density of 8.10⁵ cells/well. Plates were incubated for 4 hrs, 37° C., 5% CO2, before infection. Cells were infected either with the tk-rr-m2-COPTG19289 virus or with the tk-rr-VVTG18277 virus both expressing luciferase at MOI range of 10⁻¹ to 10⁻⁴ particles per cell. Cell viability was determined by trypan blue exclusion using a cell counter (Vi-Cell, Beckman coulter) 96 hrs post infection (D4). Quantification of the % cell survival for LOVO (FIG. 7A) and HCT116 (FIG. 7B) demonstrated that oncolytic potency provided by the m2-defective COPTG19289 virus was comparable to that obtained with the m2-positive VVTG18277 in both LOVO cells and HCT116. Specifically, LOVO cells were lysed upon infection of VVTG18277 and COPTG19289 at MOI of 10⁻¹ and 10⁻² whereas 80% cell viability was observed at a low MOI of 10⁻³ and entirely preserved at MOI of 10⁻⁴. Viral oncolytic activity was even higher in HCT116 cells since no cell viability (0%) could be detected at MOI of 10⁻¹, 10⁻² and 10⁻³ and less than 50% of cells were viable at MOI of 10⁻⁴. MOCK treatment had no effect on cells and was used to determine the 100% of viability of LOVO and HCT116 cells.

This absence of discrepancy between double and triple-deleted viruses was confirmed in other tumor cell lines including melanoma B16F10 (ATCC® CCL-6475™), murine colon carcinoma CT26WT (ATCC® CRL-2638™) and murine colon adenocarcinoma MC38WT cells (available from Kerafast and Cellosaurus CVCL_B288). Both vaccinia viruses were oncolytic in these three cell lines at MOI of 10⁻¹ and in B16F10 and MC38WT at MOI of 10⁻² while partially oncolytic in CT26WT.

In conclusion, recombinant m2-defective virus showed a comparable oncolytic activity as their m2-positive counterpart which supports the fact that impairment of M2L locus did not adversely affect oncolytic activity in tumor cell lines

Transgene Expression In Vivo

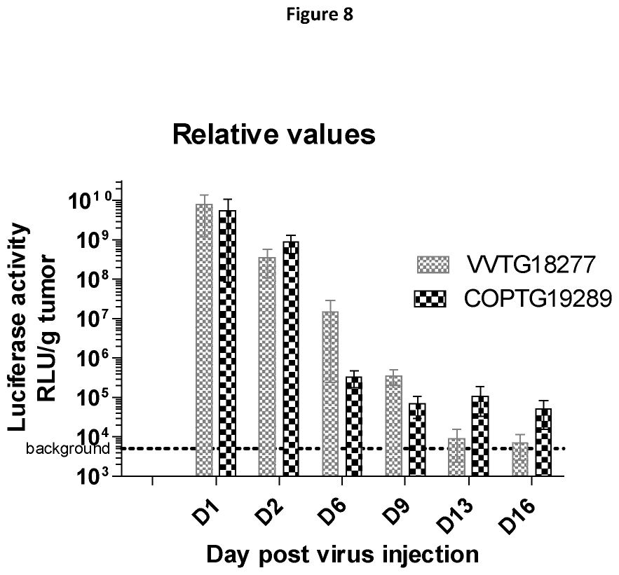

Luciferase expression generated from the tk-rr-m2-oncolytic vaccinia virus (COPTG19289) was assessed in C57BL/6 mice implanted with B16F10 tumors following subcutaneous injection and compared to the one obtained with tk-rr-VVTG18277 virus. Each virus (10⁷ pfu) was injected intratumorally at day 0, 3, 6, 10 and 14 and tumor samplings were collected at day 1, 2, 6, 9, 13 and 16 for evaluation of luciferase activity par gram of tumor (RLU/g tumor). As illustrated in FIG. 8, for both viruses, a strong luciferase activity was detected the first days (D1 and D2) following virus injection and decreased thereafter. However, luciferase expression reached the background level 13 days after infection of VVTG18277 whereas a weak but persistent expression level was measured upon infection of COPTG19289 which was maintained over time (D9, D13 and D16).

Antitumoral Activity

Antitumoral activity provided by the tk-rr-m2-oncolytic vaccinia virus (COPTG19289) was assayed in three tumor models, B16F10, CT26 and HT116, respectively.

In a first setting, C57BL/6 mice (10 mice/group) were implanted with B16F10 tumors by subcutaneous injection. When the tumors reached a volume of 25-100 mm³, the tumor of each animal was measured, and mice were randomized and injected by intratumoral route with 10⁷ pfu of COPTG19289, VVTG18277 or MOCK vehicle (negative control) at D0, D3, D6, D10 and D14. Animal survival and tumor growth were followed twice a week (mice are killed when the tumor volume reached 2000 mm³ or above). There are no drastic differences between the two VV-treated groups. Notably, several animals displayed a slowed tumor growth in both groups. In contrast, tumor growth was very rapid in Mock-treated animals reaching 2000 mm³ in 24 days, resulting in death of all mice at D24. Survival of mice was improved by the vaccinia virus treatment. Specifically, in this experiment, 50% survival was obtained at D23 for mice treated with the tk-rr-VVTG18277 and at D28 for mice treated with the tk-rr-m2-COPTG19289. For clarity, the survival curves matched between the two groups of VV, except that 2 out of the 10 injected mice died few days later (data not shown).

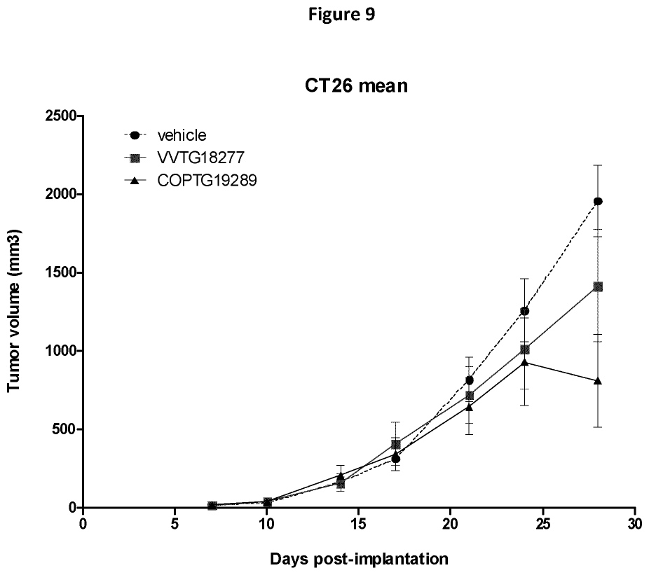

Antitumoral activity was also assayed in Balb/c mice implanted with CT26 tumors by subcutaneous injection. When the tumors reached a volume of 25-100 mm³, the tumors were individually measured, and mice were randomized (D0) before being injected intratumorally with the tk-rr-m2-oncolytic vaccinia virus (COPTG19289) or tk-rr-VVTG18277 virus or MOCK vehicle (10 mice/group). Fifty μl corresponding to 10⁷ pfu of each vaccinia virus preparation (or mock) were injected in the tumor at D0, D3, D6, D10 and D14. Tumor growth was followed twice a week and mice were killed when the tumor volume reached 2000 mm³ or above. As illustrated in FIG. 9, tumor volume in Mock-treated animals increased rapidly to reach 2000 mm³ at D28 whereas tumor growth was delayed in VV-treated groups with a tumor volume below 1000 mm³ only in tk-rr-m2-vaccinia virus.

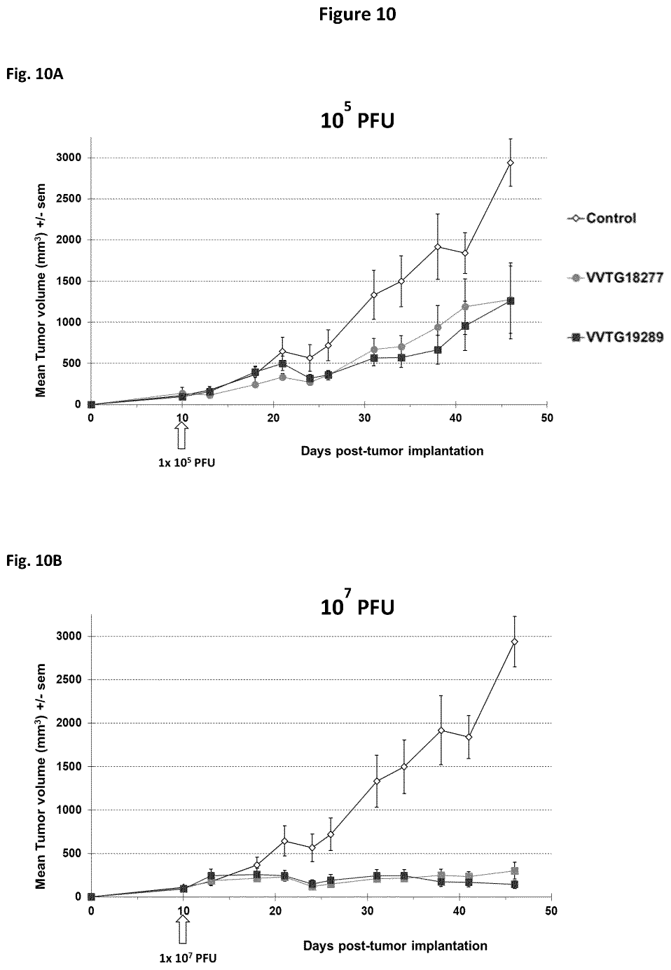

Antitumoral activity was also assayed in Swiss Nude mice (10 mice/group) implanted with HT116 tumors by subcutaneous injection. Two different doses of vaccinia viruses were injected intravenously 10 days following tumor implantation, respectively 10⁵ and 10⁷ pfu. Tumor growth was followed twice a week and mice were killed when the tumor volume reached 2000 mm³ or above. As expected, tumor volume in Mock-treated animals increased rapidly to reach 2000 mm³ or higher 45 days post tumor implantation whereas tumor growth was delayed in groups treated with 10⁵ pfu of VV. Notably, tumor growth was completely inhibited in the two groups injected with 10⁷ pfu of vaccinia virus as illustrated in FIG. 10.

In conclusion, the modification of VV M2L locus to render the vaccinia virus unable to produce the immunosuppressive M2 protein has no impact on the oncolytic activity, the antitumoral effect and the expression of the transgene.

### Example 4: Mixed Lymphocyte Reaction (MLR) Assays

The supernatants obtained from CEF cells infected with by COPTG19289 (tk-, rr- and m2-), or VVTG18058 (tk-rr-) or MVAN33 were evaluated in MLR for their ability to activate lymphocytes. Culture supernatants were harvested 48 h post-infection (MOI 0.05) and were concentrated about 20-fold.

PBMC were purified by Ficoll-Paque PLUS (GE healthcare) from blood collected from healthy donors. More specifically, 3×10⁵ PBMC from 2 different donors were mixed in 96-well microplate. The concentrated supernatants were added to the PBMC culture (20 μL in 200 μL) either undiluted or diluted 10 or 100-fold to yield a final “supernatant concentration” of 2, 0.2 and 0.02-fold, respectively, and cultured for 72 h at 37° C. in 5% CO₂ atmosphere. Addition of RPMI medium was used as a negative control. IL-2 secretion was quantified in culture supernatants by ELISA (IL-2*-2ELISA-MAX™ deluxe Set kit from BioLegend) as a marker of lymphocytes' activation. The measures were normalized by dividing the mean of IL-2 concentration of the three replicates of a given sample by the mean of IL-2 concentration of the three replicates of PBMC incubated with medium.

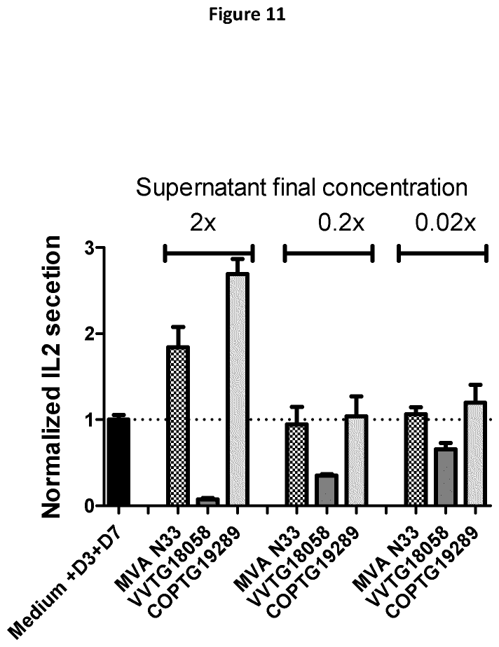

Negative control represents a normalized lymphocyte activation status of 1. As illustrated in FIG. 11, PBMC incubated in the presence of supernatants of cells infected with both MVA and COPTG19289 (tk-rr-m2-; TD) induced lymphocyte activation reaching a value close to one when diluted 10 or 100-fold and beyond 1 when tested undiluted. In marked contrast, the VVTG18058 (tk-rr-; DD)-infected supernatants showed a clear inhibition of lymphocyte activation at all dilutions tested confirming the immunosuppressive activity of the M2-encoding virus.

### Example 5: Antitumoral Activity in Humanized NCG-CD34+ Mice

Antitumoral activity provided by the m2-COPTG19289 virus was assessed in NOD/Shi-scid/IL-2Rγnull immunodeficient mouse strain (NCG) humanized with CD34+ human stem cells and engrafted with human colorectal carcinoma cells HCT-116 (5×10⁶ cells injected SC in one mouse's flank representing D0). Twelve days post implantation (D12), mice received a single IV injection of either COPTG19289 (tk-rr-m2-; TD) or its m2+ counterpart VVTG18058 (tk-rr-; DD) at doses of 10⁵ pfu or 10⁶ pfu. Vehicle-treated mice were used as negative controls. Tumor growth and mice survival were monitored over at least 60 days post cell implantation.

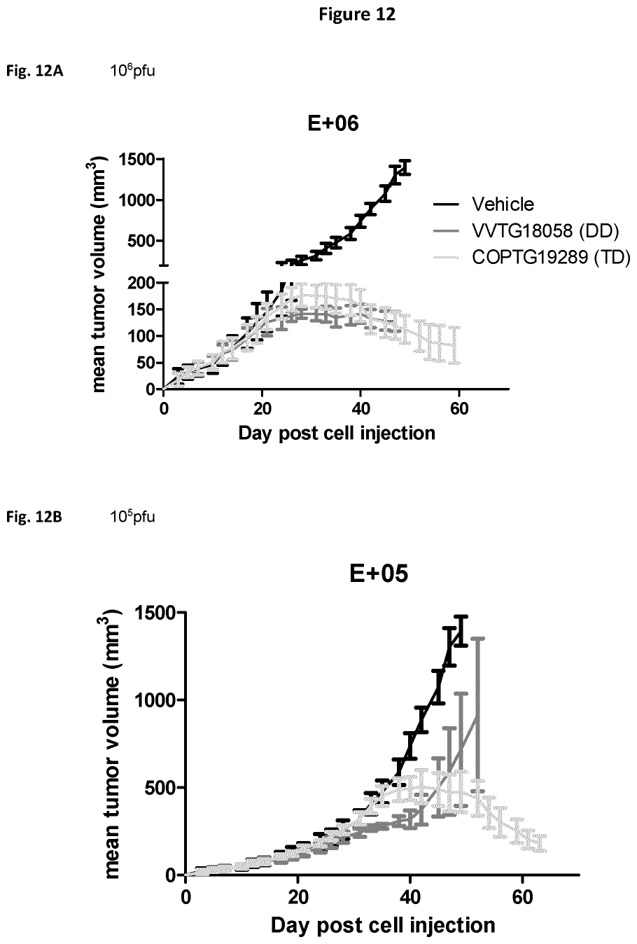

As illustrated in FIGS. 12A and B, tumor volumes increased very rapidly in the group of vehicle-treated mice. In marked contrast, tumor growth was clearly inhibited in mice treated with m2-COPTG19289 (TD) or m2+ VVTG18058 (DD) whatever the dose injected but dose-dependent toxicity issues emerged for some animals; thus, preventing the tumor growth monitoring over the 60-day period. At the 10⁶ dose, both viruses delayed tumor growth with approximately the same efficacy (FIG. 12A) but lower toxicity was observed with the TD virus COPTG19289 compared to the DD VVTG18058 virus. To be noted, that one TD-treated animal was tumor-free at 55 days post cell implantation and the tumor-free status remained over more than 85 days. At the 10⁵ dose, the TD virus COPTG19289 showed improved antitumor effect over the DD VVTG18058 virus (FIG. 12B). More specifically, tumor growth was clearly inhibited in 5/5 animals in the TD group versus 2/5 in the DD group. In addition, a reduced toxicity was observed in the TD group compared to the DD group.

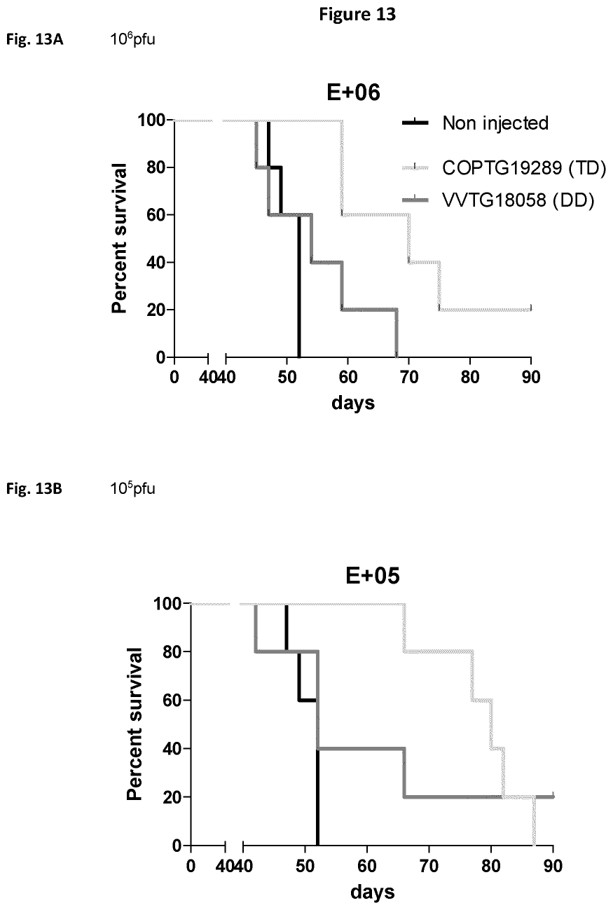

Comparison of mice survival confirmed the improved anti-tumoral effect provided by the m2-COPTG19289 (TD) compared to the m2+ VVTG18058 (DD) following a single IV injection of 10⁶ pfu (FIG. 13A) or 10⁵ pfu (FIG. 13B). More specifically, 100% of vehicle treated animals are dead in 52 days, whereas survival is clearly extended by VVTG18058 (DD) treatment and even more by COPTG19289 (TD) treatment. For example, 50% survival (FIG. 13A) was estimated at 52 days for the negative control, 54 days for the DD-10⁶ pfu-treated group and 70 days for the TD-10⁶ pfu-treated group.

Moreover, at the 10⁵ pfu doses, the 50% survival were 52 and 80 days for the DD and TD viruses respectively.

These results illustrate the improved therapeutic interest provided by m2-defective poxviruses to treat pathology such as cancers.

### REFERENCES

Antoine et al., 1998, Virology 244(2) 365-96
Baoming Liu et al., 2018, J. Virol. 92(7) e02152-17
Bloom et al., 1991, J. Virol. 65(3): 1530-42
Boyle et al., 1985, Gene 35, 169-177
Brunet et al., 1987, Nature 328: 267-70
Carpentier et al., 2003, Frontiers in Bioscience 8, e115-127
Carpentier et al., 2006, Neuro-Oncology 8(1): 60-6
Chakrabarti et al. (1997, Biotechniques 23: 1094-7
Chan, 2008, Eur. J. Immunol. 38, 2964-2968
Chaurasiya et al., 2018, Current Opinion in Immunology 51: 83-90
Chen, 2004, Nat. Rev. Immunol. 4: 336-347
Claudepierre et al., 2014, J. Virol. 88(10): 5242-55
Cole et al. in Monoclonal antibodies and Cancer Therapy; Alan Liss pp 77-96
Coligan et al., 1992 and 1994, Current Protocols in Immunology; ed J Wiley & Sons
Inc, National Institute of Health
Dariavach et al., Eur. J. Immunol. 18: 1901-5
De Graaf et al., 2018, doi.org/10.1016/j.cytogfr.2018.03.006
Erbs et al., 2008, Cancer Gene Ther. 15(1): 18-28
Evans et al. 2004, J Pharm Sci. 93:2458-75
Fallaux et al., 1998, Human Gene Ther. 9: 1909-17
Fend et al., 2014, Cancer Immunol. Res. 2, 1163-74
Foloppe et al., 2008, Gene Ther. 15: 1361-71
Gedey et al., 2006, J. Virol. 80: 8676-85
Guo et al., 1990, J. Virol. 64: 2399-2406
Guse et al., 2011, Expert Opinion Biol. Ther. 11(5):595-608
Hammond et al, 1997, J. Virol Methods 66: 135-8
Harlow and Lane, 1988, Antibodies—A laboratory manual; Cold Spring Harbor
Laboratory, Cold Spring Harbor N.Y.
Hinthong et al., 2008, Virology 373(2): 248-62
Hodge et al., 1994, Cancer Res. 54: 5552-5
Kahn et al., 2015, J. Oncol. Doi: 10.1155/2015/847383
Kleinpetter et al., 2016, OncoImmunology 5(10): e1220467
Kumar and Boyle, 1990, Virology 179: 151-8
Liu et al., 2018, J. Virol., doi/10.1128/JVI.02152-17
Mackett et al., 1984 J. of Virol., 49: 857-64
McLaughlin et al., 1996, Cancer Res. 56: 2361-67
Needleman and Wunsh. J. Mol. Biol. 48,443-453, 1970
Parato et al., 2012, Molecular Therapy 20(4): 749-58
J. R. Robinson in “Sustained and Controlled Release Drug Delivery Systems”,
ed., Marcel Dekker, Inc., New York, 1978
Russell et al., 2012, Nat. Biotechnol. 30(7): 658-70
Scott-Algara et al., 2010 PLOS One 5(1), e8761
Smith et al., 1993, Vaccine 11(1): 43-53
Smith and Kotwal, 2002, Crit. Rev. Microbiol. 28(3): 149-85
Yi, 2011, J Immunol. 186:2739-2749
Zhou et al., 2006, Blood 107, 2461-2469
WO97/20574
WO99/03885
WO00/037504
WO01/23001
WO03/053463
WO2005/042728
WO2006/085082
WO2006/93924
WO2006/108846
WO2007/056847
WO2007/113648
WO2007/123737
WO2007/147528
WO2008/114021
WO2008/129058
WO2008/138533
WO2009/53937
WO2009/065546
WO2009/065547
WO2009/100521
WO2010/130753
WO2010/130756
WO2011/066389
WO2012/001075
WO2012/122444
WO2013/022764
WO2013/079174
WO2013/173223
WO2013/181634
WO2014/053571
WO2016/008976
WO2016/149201
WO2016/196237
WO2018/122088
EP17306012.0
EP 463 756
EP 1 162 982
U.S. Pat. No. 5,250,534
U.S. Pat. No. 6,469,012
U.S. Pat. No. 6,686,152
U.S. Pat. No. 6,984,720
U.S. Pat. No. 7,108,844
U.S. Pat. No. 7,109,003
U.S. Pat. No. 7,456,009
U.S. Pat. No. 7,700,569
U.S. Pat. No. 8,017,114
U.S. Pat. No. 8,143,379
U.S. Pat. No. 8,217,149
U.S. Pat. No. 8,491,895
US2007-016108

1.-28. (canceled)

29. A modified poxvirus which genome comprises in the native (wild-type) context a M2L locus encoding a functional m2 poxviral protein and which is modified to be defective for the said m2 function; wherein said functional M2 poxviral protein is able to bind CD80 or CD86 co-stimulatory ligands or both CD80 and CD86 co-stimulatory ligands and wherein said defective m2 function is unable to bind said CD80 and CD86 co-stimulatory ligands.

30. The modified poxvirus of claim 29, wherein the modified poxvirus is generated or obtained from a Chordopoxvirinae.

31. The modified poxvirus of claim 30, wherein the modified poxvirus is a member of the Orthopoxvirus or of the Leporipoxvirus genus.

32. The modified poxvirus of claim 31, wherein the modified poxvirus is a vaccinia virus or a myxoma virus.

33. The modified poxvirus of claim 29, wherein the inability to bind said CD80 and CD86 co-stimulatory ligands originates from a genetic lesion within the M2L locus or from an abnormal interaction impairing the m2 function either directly or indirectly.

34. The modified poxvirus of claim 33, wherein said genetic lesion(s) include partial or total deletion of the M2L locus and/or one or more non-silent mutation(s) either within the m2-coding sequence or in the regulatory elements controlling M2L expression.

35. The modified poxvirus of claim 29, wherein the modified poxvirus is further modified in a region other than M2L locus.

36. The modified poxvirus of claim 35, wherein the modified poxvirus is further modified in any locus/loci selected from the group consisting of:
the J2R locus, resulting in a modified poxvirus defective for both m2 and tk functions;
the I4L and/or F4L locus/loci, resulting in a modified poxvirus defective for both m2 and rr functions;
the J2R and I4L/F4L loci, resulting in a modified poxvirus defective for m2, tk and rr activities; and
any combination thereof.

37. The modified poxvirus of claim 29, wherein the modified poxvirus is oncolytic and/or recombinant.

38. The modified poxvirus of claim 37, wherein the modified poxvirus is recombinant and is engineered to express at least one polypeptide selected from the group consisting of antigenic polypeptides, polypeptides having nucleotide pool modulating function, and immunomodulatory polypeptides.

39. The modified poxvirus of claim 38, wherein said immunomodulatory polypeptide is selected from the group consisting of cytokines, chemokines, ligands, antibodies, and any combination thereof.

40. The modified poxvirus of claim 39, wherein said antibody specifically binds an immune checkpoint protein.

41. The modified poxvirus of claim 40, wherein the modified poxvirus expresses an antagonist antibody that specifically binds to PD-L1 or CTLA4.

42. The modified poxvirus of claim 41, wherein the modified poxvirus is selected from the group consisting of a poxvirus defective for m2, tk, and rr activities and encoding an anti-CTLA-4 antibody, and a poxvirus defective for m2, tk, and rr activities and encoding an anti-PD-L1 antibody.

43. A method for producing the modified poxvirus of claim 29 comprising the steps of a) preparing a producer cell line, b) transfecting or infecting the prepared producer cell line with the modified poxvirus, c) culturing the transfected or infected producer cell line under suitable conditions so as to allow the production of the virus, d) recovering the produced virus from the culture of said producer cell line and optionally e) purifying said recovered virus.

44. A composition comprising a therapeutically effective amount of the modified poxvirus claim 29 and a pharmaceutically acceptable vehicle.

45. The composition of claim 44, comprising from approximately 10³ to approximately 10¹² pfu of the modified poxvirus.

46. The composition of claim 44, which is formulated for intravenous or intratumoral administration.

47. A method for treating or preventing a proliferative disease selected from the group consisting of cancers as well as diseases associated to an increased osteoclast activity and cardiovascular diseases, comprising administering the composition of claim 44 in a subject in need thereof.

48. The method of claim 47, wherein said cancer is selected from the group consisting of renal cancer, prostate cancer, breast cancer, colorectal cancer, lung cancer, liver cancer, gastric cancer, bile duct carcinoma, endometrial cancer, pancreatic cancer, and ovarian cancer; and/or wherein said disease associated with an increased osteoclast activity is selected from the group consisting of rheumatoid arthritis and osteoporosis; and/or wherein said cardiovascular diseases is restenosis.

49. A method for stimulating or improving an immune response, comprising administering the composition of claim 44 in a subject in need thereof.

50. The method of claim 47, for use as stand-alone therapy or in conjunction with one or more additional therapies.
